(19) United States
(12) Patent Application Publication
(10) Pub. No.: US 2009/0253752 A1
(43) Pub. Date: Oct. 8, 2009

(54) COMBINATION OF DPP-IV INHIBITOR, PPAR ANTIDIABETIC AND METMORFIN

(76) Inventors: Bryan Burkey, Winchester, MA (US); Thomas Edward Hughes, Concord, MA (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/577,941

(22) PCT Filed: Oct. 21, 2005
(86) PCT No.: PCT/US05/37819

§ 371 Date: Apr. 25, 2007

Related U.S. Application Data
(60) Provisional application No. 60/621,891, filed on Oct. 25, 2004.

(51) Int. Cl. A61K 31/4439; A61K 31/427; A61K 31/40; A61K 31/155

(52) U.S. Cl. 514/342; 514/369; 514/408; 514/635

(57) ABSTRACT

The invention relates to a combination, such as a combined preparation or pharmaceutical composition, respectively, which comprises;
1) a dipeptidylpeptidase-IV (DPP-IV) inhibitor,
2) one antidiabetic selected from thiazolidinediones (glitazones), non-glitazone type PPARγ agonists, PPARα agonists or dual PPARγ/PPARα agonists, and
3) metformin,
   for simultaneous, separate or sequential use, especially in the prevention, delay of progression or treatment of conditions mediated by dipeptidylpeptidase-IV (DPP-IV), in particular diabetes, more particular type 2 diabetes mellitus, conditions of impaired glucose tolerance (IGT), conditions of impaired fasting plasma glucose, metabolic acidosis, ketosis, arthritis, obesity and osteoporosis; the use of such combination for the preparation of a pharmaceutical preparation for the prevention, delay of progression or treatment of such conditions; the use of such combination for the cosmetic treatment of a mammal in order to effect a cosmetically beneficial loss of body weight; a method of prevention, delay of progression or treatment of conditions mediated by DPP-IV; a method of improving the bodily appearance of a warm-blooded animal.

The invention relates to a combination, particularly a pharmaceutical combination, such as a combined preparation or pharmaceutical composition, respectively, which comprises i) a dipeptidylpeptidase-IV (DPP-IV) inhibitor, ii) one antidiabetic selected from thiazolidinediones (glitazones), non-glitazone type PPARγ agonists, PPARα agonists or dual PPARγ/PPARα agonists, and iii) metformin, for simultaneous, separate or sequential use, especially in the prevention, delay of progression or treatment of conditions mediated by dipeptidylpeptidase-IV (DPP-IV), in particular diabetes, more particular type 2 diabetes mellitus, conditions of impaired glucose tolerance (IGT), conditions of impaired fasting plasma glucose, metabolic acidosis, ketosis, arthritis, obesity and osteoporosis; the use of such combination for the preparation of a pharmaceutical preparation for the prevention, delay of progression or treatment of such conditions; the use of such combination for the cosmetic treatment of a mammal in order to effect a cosmetically beneficial loss of body weight; a method of prevention, delay of progression or treatment of conditions mediated by DPP-IV; a method of improving the bodily appearance of a warm-blooded animal.

PPAR ANTIDIABETIC: The antidiabetic selected from thiazolidinediones (glitazones), non-glitazone type PPARγ agonists, PPARα agonists or dual PPARγ/PPARα agonists will be referred hereinafter as “PPAR ANTIDIABETIC”.

DPP-IV is responsible for inactivating GLP-1. More particularly, DPP-IV generates a GLP-1 receptor antagonist and thereby shortens the physiological response to GLP-1. GLP-1 is a major stimulator of pancreatic insulin secretion and has direct beneficial effects on glucose disposal.

Non-insulin dependent diabetes mellitus (type 2 diabetes mellitus) is characterized by both increased peripheral insulin resistance and abnormal insulin secretion. At least three abnormalities of insulin secretion are recognized: in the first phase, insulin secretion is lost and in the second phase insulin is both delayed and inadequate in the face of elevated circulating glucose levels. Several metabolic, hormonal, and pharmacological entities are known to stimulate insulin secretion including glucose, amino-acids and gastrointestinal peptides. The Diabetes Control and Complications Trial (DCCT) has established that lowering of blood glucose is associated with decreases in the onset and progression of diabetic microvascular complications (Diabetes Control and Complications Trial Research Group; N. Engl. J. Med. 1993, 329, 977-986). IGT is an impairment of glucose homeostasis closely related to type 2 diabetes mellitus. Both conditions convey a great risk of macrovascular disease. Therefore, one therapeutic focus is on optimizing and potentially normalizing glycemic control in subjects with type 2 diabetes mellitus, conditions of impaired fasting plasma glucose, or IGT. Presently available agents need to be improved in order to better meet this therapeutic challenge.

The present invention relates to a combination which comprises 1) a dipeptidylpeptidase-IV (DPP-IV) inhibitor, 2) one PPAR ANTIDIABETIC and 3) metformin, or in each case the pharmaceutically acceptable salt of such a compound and optionally at least one pharmaceutically acceptable carrier; for simultaneous, separate or sequential use. This combination will hereinafter be designated as COMBINATION OF THE INVENTION.

In the present context “a DPP-IV inhibitor” is also intended to comprise active metabolites and prodrugs thereof, such as active metabolites and prodrugs of DPP-IV inhibitors. A “metabolite” is an active derivative of a DPP-IV inhibitor produced when the DPP-IV inhibitor is metabolised. A “prodrug” is a compound that is either metabolised to a DPP-IV inhibitor or is metabolised to the same metabolite(s) as a DPP-IV inhibitor.

DPP-IV inhibitors are known in the art. For example, DPP-IV inhibitors are in each case generically and specifically disclosed e.g. in WO 98/19998, DE19616 486 A1, WO 00/34241, WO 95/15309, WO 01/72290, WO01/52825, WO 9310127, WO 9925719, WO 9938501, WO 9946272, WO 9967278 and WO 9967279.

Preferred DPP-IV inhibitors are described in the following patent applications; WO 02053548 especially compounds 1001 to 1293 and examples 1 to 124, WO 02067918 especially compounds 1000 to 1278 and 2001 to 2159, WO 02066627 especially the described examples, WO 02/068420 especially all the compounds specifically listed in the examples I to LXIII and the described corresponding analogues, even preferred compounds are 2(28), 2(88), 2(119), 2(136) described in the table reporting IC50, WO 02083128 especially examples 1 to 13, US 2003096846 especially the specifically described compounds, WO 2004/037181 especially examples 1 to 33, WO 0168603 especially compounds of examples 1 to 109, EP1258480 especially compounds of examples 1 to 60, WO 0181337 especially examples 1 to 118, WO 02083109 especially examples 1A to 1D, WO 030003250 especially compounds of examples 1 to 166, most preferably 1 to 8, WO 03035067 especially the compounds described in the examples, WO 03/035057 especially the compounds described in the examples, US2003216450 especially examples 1 to 450, WO 99/46272 especially compounds of claims 12, 14, 15 and 17, WO 0197808 especially compounds of claim 2, WO 03002553 especially compounds of examples 1 to 33, WO 01/34594 especially the compounds described in the examples 1 to 4, WO 02051836 especially examples 1 to 712, EP1245568 especially examples 1 to 7, EP1258476 especially examples 1 to 32, US 2003087950 especially the described examples, WO 02/076450 especially examples 1 to 128, WO 03000180 especially examples 1 to 162, WO 03000181 especially examples 1 to 66, WO 03004498 especially examples 1 to 33, WO 0302942 especially examples 1 to 68, U.S. Pat. No. 6,482,844 especially the described examples, WO 0155105 especially the compounds listed in the examples 1 and 2, WO 0202560 especially examples 1 to 166, WO 03004496 especially examples 1 to 103, WO 03/024965 especially examples 1 to 54, WO 0303727 especially examples 1 to 209, WO 0368757 especially examples 1 to 88, WO 03074500 especially examples 1 to 72, examples 4.1 to 4.23, examples 5.1 to 5.10, examples 6.1 to 6.30, examples 7.1 to 7.23, examples 8.1 to 8.10, examples 9.1 to 9.30, WO 02038541 especially examples 1 to 53, WO 02062764 especially examples 1 to 293, preferably the compound of example 95 (2-{{3-(Aminomethyl)-4-butoxy-2-neopentyl-1-oxo-1,2 dihydro-6-isoquinolinyl}oxy}acetamide hydrochloride), WO 0230890 especially examples 1-1 to 1-109, examples 2-1 to 2-9, example 3, examples 4-1 to 4-19, examples 5-1 to 5-39, examples 6-1 to 6-4, examples 7-1 to 7-10, examples 8-1 to 8-8, examples 7-1 to 7-7 of page 90, examples 8-1 to 8-59 of pages 91 to 95, examples 9-1 to 9-33, examples 10-1 to 10-20, US 2003225102 especially compounds 1 to 115, compounds of examples 1 to 121, preferably compounds a) to z), aa) to az), ba) to bz), ca) to cz) and da) to dk), WO 0214271 especially examples 1 to 320 and US 2003096857, WO 2004/052850 especially the specifically described compounds such as examples 1 to 42 and compounds of claim 1, DE 102 56 264 A1 especially the described compounds such as examples 1 to 181 and the compounds of claim 5, WO 04/076433 especially the compounds specifically described, such as listed in table A, preferably the compounds listed in table B, preferably compounds I to XXXXVII, or compounds of claims 6 to 49, WO 04/071454 especially the specifically described compounds e.g. compounds I to 53 or compounds of tables Ia to If, or compounds of claims 2 to 55, WO 02/068420 especially the compounds specifically described, such as the compounds I to LXIII or Beispiele I and analogues 1 to 140 or Beispiele 2 and analogues 1 to 174 or Beispiele 3 and analogues 1, or Beispiele 4 to 5, or Beispiele 6 and analogues 1 to 5, or Beispiele 7 and analogues 1-3, or Beispiele 8 and analogue 1, or Beispiele 9, or Beispiele 10 and analogues 1 to 531 even preferred are compounds of claim 13, WO 03/000250 especially the compounds specifically described, such as the compounds I to 166, preferably compounds of examples 1 to 9, WO 03/024942 especially the compounds specifically described, such compounds I to 59, compounds of table 1 (1 to 68), compounds of claims 6, 7, 8, 9, WO 03024965024942 especially the compounds specifically described, such compounds I to 54, WO03002593 especially the compounds specifically described, such compounds table 1 or of claims 2 to 15, WO03037327 especially the compounds specifically described, such compounds of examples 1 to 209 WO 03/000250 especially the compounds specifically described, such as the compounds I to 166, preferably compounds of examples 1 to 9, WO 03/024942 especially the compounds specifically described, such compounds I to 59, compounds of table 1 (1 to 68), compounds of claims 6, 7, 8, 9, WO 03024965024942 especially the compounds specifically described, such compounds I to 54, WO03002593 especially the compounds specifically described, such compounds table 1 or of claims 2 to 15, WO03037327 especially the compounds specifically described, such compounds of examples 1 to 209, WO0238541, WO0230890.

WO 03/000250 especially the compounds specifically described, such as the compounds I to 166, preferably compounds of examples 1 to 9, WO 03/024942 especially the compounds specifically described, such compounds I to 59, compounds of table 1 (1 to 68), compounds of claims 6, 7, 8, 9, WO 03024965 especially the compounds specifically described, such compounds I to 54, WoO3002593 especially the compounds specifically described, such compounds table 1 or of claims 2 to 15, WO03037327 especially the compounds specifically described, such compounds of examples 1 to 209, WO0238541 especially the compounds specifically described, such compounds of examples 1 to 53.

In each case in particular in the compound claims and the final products of the working examples, the subject matter of the final products, the pharmaceutical preparations and the claims are hereby incorporated into the present application by reference to these publications.

The DPP-IV inhibitor can be peptidic or non-peptidic. Preferably, the DPP-IV inhibitor is non-peptidic.

DPP-IV inhibitors are in each case generically and specifically disclosed in WO 98/19998, DE 196 16 486 A1, WO 00/34241 and WO 95/15309, in each case in particular in the compound claims and the final products of the working examples, the subject-matter of the final products, the pharmaceutical preparations and the claims are hereby incorporated into the present application by reference to these publications. DPP728 and LAF237 are specifically disclosed in Example 3 of WO 98/19998 and Example 1 of WO 00/34241, respectively. A DPP-IV inhibitor of formula VI (see above) is specifically described in Diabetes 1998, 47, 1253-1258. DPP728 can be formulated as described on page 20 of WO 98/19998.

In a further preferred embodiment, the DPP-IV inhibitor is a N-peptidyl-O-aroyl hydroxylamine or a pharmaceutically acceptable salt thereof. Aroyl is, for example, naphthylcarbonyl; or benzoyl which is unsubstituted or mono- or disubstituted, for example, by lower alkoxy, lower alkyl, halogen or, preferably, nitro. The peptidyl moiety comprises preferably two α-amino acids, e.g. glycine, alanine, leucine, phenylalanine, lysine or proline, of which the one attached directly to the hydroxylamine nitrogen atom is preferably proline.

WO 9819998 discloses N—(N′-substituted glycyl)-2-cyano pyrrolidines, preferred compounds are described in the examples 1 to 66 and claims 2 to 5 especially claim 5, in particular 1-[2-[5-Cyanopyridin-2-yl]amino]-ethylamino]acetyl-2-cyano-(S)-pyrrolidine (DPP728).

Preferred compounds described in WO03/002553 are listed on pages 9 to 11 and are incorporated into the present application by reference.

DE19616 486 A1 discloses val-pyr, val-thiazolidide, isoleucyl-thiazolidide, isoleucyl-pyrrolidide, and fumar salts of isoleucyl-thiazolidide and isoleucyl-pyrrolidide.

Published patent application WO 0034241 and published U.S. Pat. No. 6,110,949 disclose N-substituted adamantyl-amino-acetyl-2-cyano pyrrolidines and N-(substituted glycyl)-4-cyano pyrrolidines respectively. DPP-IV inhibitors of interest are specially those cited in claims 1 to 4. In particular these applications describe the compound I-[[(3-Hydroxy-1-adamantyl)amino]acetyl]-2-cyano-(S)-pyrrolidine (also known as LAF237).

WO 9515309 discloses amino acid 2-cyanopyrrolidine amides as inhibitors of DPP-IV and WO 9529691 discloses peptidyl derivates of diesters of alpha-aminoalkylphosphonic acids, particularly those with proline or related structures. DPP-IV inhibitors of interest are specially those cited in Table 1 to 8.

In WO 01/72290 DPP-IV inhibitors of interest are specially those cited in example 1 and claims 1, 4, and 6.

WO01/52825 also discloses (S)-1-{2-[5-cyanopyridin-2-yl)amino]ethyl-aminoacetyl)-2-cyano-pyrrolidine or (S)-1-[(3-hydroxy-1 adamantyl)amino]acetyl-2-cyano-pyrrolidine (LAF237).

WO 9310127 discloses proline boronic esters useful as DPP-IV inhibitors. DPP-IV inhibitors of interest are specially those cited in examples 1 to 19.

Published patent application WO 9925719 discloses sulphostin, a DPP-IV inhibitor prepared by culturing a Streptomyces microorganism.

WO 9938501 discloses N-substituted 4- to 8-membered heterocyclic rings. DPP-IV inhibitors of interest are specially those cited in claims 15 to 20.

WO 9946272 discloses phosphoric compounds as inhibitors of DPP-IV. DPP-IV inhibitors of interest are specially those cited in claims 1 to 23.

Other preferred DPP-IV inhibitors are the compounds of formula I, II or III disclosed in the patent application WO 03/057200 on page 14 to 27. Most preferred DPP-IV inhibitors are the compounds specifically described on pages 28 and 29.

Published patent applications WO 9967278 and WO 9967279 disclose DPP-IV prodrugs and inhibitors of the form A-B-C where C is either a stable or unstable inhibitor of DPP-IV.

Preferably, the N-peptidyl-O-aroyl hydroxylamine is a compound of formula VII

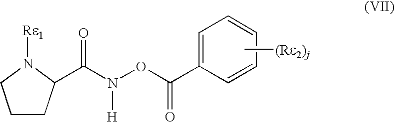

wherein
j is 0, 1 or 2;
R_{ε}_{₁} represents the side chain of a natural amino acid; and
R_{ε}_{₂} represents lower alkoxy, lower alkyl halogen or nitro;
or a pharmaceutically acceptable salt thereof.

In a very preferred embodiment of the invention, the N-peptidyl-O-aroyl hydroxylamine is a compound of formula VIIa

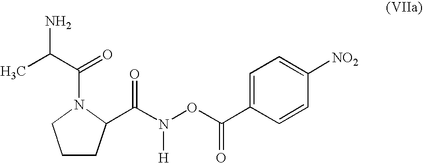

or a pharmaceutically acceptable salt thereof.

N-Peptidyl-O-aroyl hydroxylamines, e.g. of formula VII or VIIa, and their preparation are described by H. U. Demuth et al. in J. Enzyme Inhibition 1988, Vol. 2, pages 129-142, especially on pages 130-132.

Preferred DPP-IV inhibitors are those described by Mona Patel and col. (Expert Opinion Investig Drugs. 2003 April; 12(4):623-33) on the paragraph 5, especially P32/98, K-364, FE-999011, BDPX, NVP-DDP-728 and others, which publication is hereby incorporated by reference especially the described DPP-IV inhibitors.

Preferred DPP-IV inhibitors are N-substituted adamantyl-amino-acetyl-2-cyano pyrrolidines, N (substituted glycyl)-4-cyano pyrrolidines, N—(N′-substituted glycyl)-2-cyanopyrrolidines, N-aminoacyl thiazolidines, N-aminoacyl pyrrolidines, L-allo-isoleucyl thiazolidine, L-threo-isoleucyl pyrrolidine, and L-allo-isoleucyl pyrrolidine, 1-[2-[(5-cyanopyridin-2-yl)amino]ethylamino]acetyl-2-cyano-(S)-pyrrolidine and pharmaceutical salts thereof.

Preferred DPP-IV inhibitors are those described by Mona Patel and col. (Expert Opinion Investig Drugs. 2003 April; 12(4):623-33) on the paragraph 5, especially P32/98, K-364, FE-999011, BDPX, NVP-DDP-728 and others, which publication is hereby incorporated by reference especially the described DPP-IV inhibitors.

Another preferred inhibitor is the compound BMS-477118 disclosed in WO 2001068603 or U.S. Pat. No. 6,395,767 (compound of example 60) also known as is (1S,3S,5S)-2-[(2S)-2-amino-2-(3-hydroxytricyclo[3.3.1.1^{3,7}]dec-1-yl)-1-oxoethyl]-2-azabicyclo[3.1.0]hexane-3-carbonitrile, benzoate (1:1) as depicted in Formula M of the patent application WO 2004/052850 on page 2, and the corresponding free base, (1S,3S,5S)-2-[(2S)-2-amino-2-(3-hydroxy-tricyclo[3.3.1.1^{3,7}]dec-1-yl)-1-oxoethyl]-2-azabicyclo-[3.1.0]hexane-3-carbonitrile (M′) and its monohydrate (M″) as depicted in Formula M of the patent application WO 2004/052850 on page 3. The compound BMS-477118 is also known as saxagliptin.

Another preferred inhibitor is the compound GSK23A disclosed in WO 03/002531 (example 9) also known as (2S,4S)-1-((2R)-2-Amino-3-[(4-methoxybenzyl)sulfonyl]-3-methylbutanoyl)-4-fluoropyrrolidine-2-carbonitrile hydrochloride.

FE-999011 is described in the patent application WO 95/15309 page 14, as compound No. 18.

Other very preferred DPP-IV inhibitors of the invention are described in the International patent application WO 02/076450 (especially the examples 1 to 128) and by Wallace T. Ashton (Bioorganic & Medicinal Chemistry Letters 14 (2004) 859-863) especially the compound I and the compounds listed in the tables 1 and 2. The preferred compound is the compound 21e (table 1) of formula

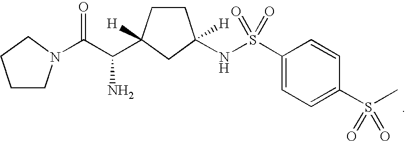

P32/98 or P3298 (CAS number: 251572-86-8) also known as 3-[(2S,3S)-2-amino-3-methyl-1-oxopentyl]thiazolidine can be used as 3-[(2S,3S)-2-amino-3-methyl-1-oxopentyl]thiazolidine and (2E)-2-butenedioate (2:1) mixture such as shown below

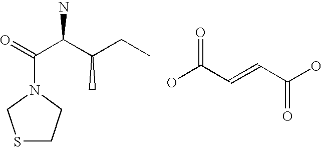

and is described in WO 99/61431 and also in Diabetes 1998, 47, 1253-1258, in the name of Probiodrug, as well as the compound P93/01 described by the same company.

Other preferred DPP-IV inhibitors are the compounds disclosed in the patent application WO 02/083128 such as in the claims 1 to 5. Most preferred DPP-IV inhibitors are the compounds specifically described by the examples 1 to 13 and the claims 6 to 10.

Other preferred DPP-IV inhibitors are described in the patent applications WO 2004/037169 especially those described in the examples 1 to 48 and WO 02/062764 especially the described examples 1 to 293, even preferred are the compounds 3-(aminomethyl)-2-isobuthyl-1-oxo-4-phenyl-1,2-dihydro-6-isoquinolinecarboxamide and 2-{[3-(aminomethyl)-2-isobuthyl-4-phenyl-1-oxo-1,2-dihydro-6-isoquinolyl]oxy}acetamide described on page 7 and also in the patent application WO2004/024184 especially in the reference examples 1 to 4.

Other preferred DPP-IV inhibitors are described in the patent application WO 03/004498 especially examples 1 to 33 and most preferably the compound of the formula

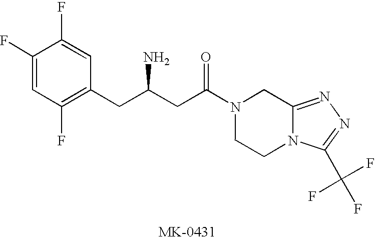

described by the example 7 and also known as MK-0431 or Sitagliptin.

Preferred DPP-IV inhibitors are also described in the patent application WO 2004/037181 especially examples 1 to 33, most preferably the compounds described in the claims 3 to 5. Preferred DPP-IV inhibitors are N-substituted adamantyl-amino-acetyl-2-cyano pyrrolidines, N (substituted glycyl)-4-cyano pyrrolidines, N—(N′-substituted glycyl)-2-cyanopyrrolidines, N-aminoacyl thiazolidines, N-aminoacyl pyrrolidines, L-allo-isoleucyl thiazolidine, L-threo-isoleucyl pyrrolidine, and L-allo-isoleucyl pyrrolidine, 1-[2-[(5-cyanopyridin-2-yl)amino]ethylamino]acetyl-2-cyano-(S)-pyrrolidine, MK-431 and pharmaceutical salts thereof.

Most preferred DPP-IV inhibitors are selected from [S]-1-[2-(5-cyano-2-pyridinylamino)ethylamino]acetyl-2-pyrrolidine carbonitrile monohydrochloride, (S)-1-[(3-hydroxy-1-adamantyl)amino]acetyl-2-cyano-pyrrolidine and L-threo-isoleucyl thiazolidine (compound code according to Probiodrug: P32/98 as described above), MK-0431, 3-(aminomethyl)-2-isobuthyl-1-oxo-4-phenyl-1,2-dihydro-6-isoquinolinecarboxamide and 2-{[3-(aminomethyl)-2-isobuthyl-4-phenyl-1-oxo-1,2-dihydro-6-isoquinolyl]oxy}acetamide and optionally pharmaceutical salts thereof.

Especially preferred are 1-{2-[(5-cyanopyridin-2-yl)amino]ethylamino}acetyl-2 (S)-cyano-pyrrolidine dihydrochloride (DPP728) (also named [S]-1-[2-(5-cyano-2-pyridinylamino)ethylamino]acetyl-2-pyrrolidine carbonitrile monohydrochloride), of formula

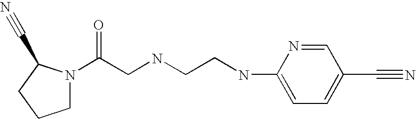

especially the dihydrochloride and monohydrochloride thereof,
and 1-[(3-hydroxy-1-adamantyl)amino]acetyl-2-cyano-, (S) (also named (S)-1-[(3-hydroxy-1-adamantyl)amino]acetyl-2-cyano-pyrrolidine, LAF237 or vildagliptin) of formula

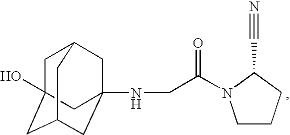

and L-threo-isoleucyl thiazolidine (compound code according to Probiodrug: P32/98 as described above), MK-0431, GSK23A, saxagliptin, 3-(aminomethyl)-2-isobuthyl-1-oxo-4-phenyl-1,2-dihydro-6-isoquinolinecarboxamide and 2-{[3-(aminomethyl)-2-isobuthyl-4-phenyl-1-oxo-1,2-dihydro-6-isoquinolyl]oxy}acetamide and optionally pharmaceutical salts thereof.

DPP728 and vildagliptin are specifically disclosed in Example 3 of WO 98/19998 and Example 1 of WO 00/34241, respectively. The DPP-IV inhibitor P32/98 (see above) is specifically described in Diabetes 1998, 47, 1253-1258. DPP728 and LAF237 can be formulated as described on page 20 of WO 98/19998 or in WO 00/34241 or in the International Patent Application No. EP2005/000400 (application number).

Especially preferred are orally active DPP-IV inhibitors. In a further embodiment, preferred DPP-IV inhibitors are preferably not dipeptidic compounds and derivatives.

Any of the substances disclosed in the above mentioned patent documents, hereby included by reference, are considered potentially useful as DPP-IV inhibitors to be used in carrying out the present invention.

DPP-IV inhibitor to be used alone according to the present invention can be used in association with a carrier.

A carrier in the instant context is a tool (natural, synthetic, peptidic, non-peptidic) for example a protein which transports specific substances through the cell membrane in which it is embedded and into the cell. Different carriers (natural, synthetic, peptidic, non-peptidic) are required to transport different substances, as each one is designed to recognize only one substance, or group of similar substances.

Any means of detection known by the person skilled in the art can be used to detect the association of the DPP-IV with a carrier, for example, by labelling the carrier.

The DPP-IV inhibitor can be a peptidic or, preferably, non-peptidic one.

Most preferred are orally active DPP-IV inhibitors and pharmaceutical salts thereof.

The active ingredients or pharmaceutically acceptable salts thereof according to the present invention may also be used in form of a solvate, such as a hydrate or including other solvents, used for crystallization.

The doses of DPP-IV inhibitor to be administered to warm-blooded animals, for example human beings, of, for example, approximately 70 kg body weight, especially the doses effective in the inhibition of the DPP-IV enzyme, e.g. in lowering blood pressure and/or in improving the symptoms of glaucoma, are from approximately 3 mg to approximately 3 g, preferably from approximately 10 mg to approximately 1 g, for example approximately from 20 mg to 200 mg, per person per day, divided preferably into 1 to 4 single doses which may, for example, be of the same size. Usually, children receive about half of the adult dose. The dose necessary for each individual can be monitored, for example by measuring the serum concentration of the active ingredient, and adjusted to an optimum level. Single doses comprise, for example, 10, 40 or 100 mg per adult patient.

The dosage of (S)-1-[(3-hydroxy-1-adamantyl)amino]acetyl-2-cyano-pyrrolidine is preferably between 10 and 150 mg daily, most preferably between 25 and 100 mg or 25 and 50 mg or 25 to 100 mg daily. Preferred examples of daily oral dosage are 25, 30, 35, 45, 50, 55, 60, 70, 80, 90, or 100 mg. The application of the active ingredient may occur up to three times a day, preferably one or two times a day.

Metformin has been widely prescribed for lowering blood glucose in patients with NIDDM and is marketed in 500, 750, 850 and 1000 mg strengths. However, because it is a short acting drug, metformin requires twice-daily or three-times-daily dosing (500-850 mg tab 2-3/day or 1000 mg bid with meals). The biguanide antihyperglycemic agent metformin disclosed in U.S. Pat. No. 3,174,901 is currently marketed in the U.S. in the form of its hydrochloride salt (Glucophage@), Bristol-Myers Squibb Company). The preparation of metformin (dimethyldiguanide) and its hydrochloride salt is state of the art and was disclosed first by Emil A. Werner and James Bell, J. Chem. Soc. 121, 1922, 1790-1794. Metformin, can be administered e.g. in the form as marketed under the trademarks GLUCOPHAGE™.

Mefformin, increases the sensitivity to insulin in peripheral tissues of the hosts. Mefformin is also involved in inhibition of glucose absorption from the intestine, suppression of hepatic gluconeogenesis, and inhibition of fatty acid oxidation. Suitable dosage regimens of Mefformin include unit doses of 500 mg two to three time's daily and can even be build up to five times daily or 850 mg once or twice daily. [Martindale, The Complete Drug Reference.

Certain controlled or sustained release formulations that employ antihyperglycemic drugs such as metformin hydrochloride have been limited to the use of an expanding or gelling agent to control the release of the drug from the dosage form. This research is exemplified by the teachings of WO 96/08243 and by the GLUCOPHAGE XR product insert which is a controlled release metformin product commercially available from Bristol-Myers Squibb. GLUCOPHAGE (metformin hydrochloride tablets) should be given in divided doses with meals while GLUCOPHAGE XR (metformin hydrochloride extended-release tablets) should generally be given once daily with the evening meal. Metformin is preferably in the form of metformin HCl.

The term “metformin” as employed herein refers to metformin or a pharmaceutically acceptable salt thereof such as the hydrochloride salt, the metformin (2:1) fumarate salt, and the metformin (2:1) succinate salt as disclosed in U.S. application Ser. No. 09/262,526 filed Mar. 4, 1999, the hydrobromide salt, the p-chlorophenoxy acetate or the embonate, and other known metformin salts of mono and dibasic carboxylic acids including those disclosed in U.S. Pat. No. 3,174,901, all of which salts are collectively referred to as metformin. It is preferred that the metformin employed herein be the metformin hydrochloride salt, namely, that marketed as GLUCOPHAGE-D or GLUCOPHAGE XR (trademark of Bristol-Myers Squibb Company).

In the present context “a DPP-IV inhibitor”, “metformin”, “a glitazone”, or any specific glitazone like “pioglitazone”, “rosiglitazone”, is also intended to comprise any pharmaceutically acceptable salt thereof, crystal form, hydrate, solvate, diastereoisomer or enantiomer thereof.

The PPAR ANTIDIABETIC is selected is selected from thiazolidinediones (glitazones), non-glitazone type PPARγ agonists, PPARα agonists or dual PPARγ/PPARα agonists.

The antidiabetic thiazolidinedione (glitazone) is, for example, (S)-((3,4-dihydro-2-(phenyl-methyl)-2H-1-benzopyran-6-yl)methyl-thiazolidine-2,4-dione (englitazone), 5-{[4-(3-(5-methyl-2-phenyl-4-oxazolyl)-1-oxopropyl)-phenyl]-methyl}-thiazolidine-2,4-dione (darglitazone), 5-{[4-(1-methyl-cyclohexyl)methoxy)-phenyl]methyl}-thiazolidine-2,4-dione (ciglitazone), 5-{[4-(2-(1-indolyl)ethoxy)phenyl]methyl}-thiazolidine-2,4-dione (DRF2189), 5-{4-[2-(5-methyl-2-phenyl-4-oxazolyl)-ethoxy)]benzyl}-thiazolidine-2,4-dione (BM-13.1246), 5-(2-naphthylsulfonyl)-thiazolidine-2,4-dione (AY-31637), bis{4-[(2,4-dioxo-5-thiazolidinyl)-methyl]phenyl}methane (YM268), 5-{4-[2-(5-methyl-2-phenyl-4-oxazolyl)-2-hydroxyethoxy]benzyl}-thiazolidine-2,4-dione (AD-5075), 5-[4-(1-phenyl-1-cyclopropanecarbonylamino)-benzyl]-thiazolidine-2,4-dione (DN-108) 5-{[4-(2-(2,3-dihydroindol-1-yl)ethoxy)phenylmethyl}-thiazolidine-2,4-dione, 5-[3-(4-chloro-phenyl])-2-propynyl]-5-phenylsulfonyl)thiazolidine-2,4-dione, 5-[3-(4-chloro-phenyl])-2-propynyl]-5-(4-fluorophenyl-sulfonyl)thiazolidine-2,4-dione, 5-{[4-(2-(methyl-2-pyridinyl-amino)-ethoxy)phenyl]methyl}-thiazolidine-2,4-dione (rosiglitazone), 5-{[4-(2-(5-ethyl-2-pyridyl)ethoxy)phenyl]-methyl}thiazolidine-2,4-dione (pioglitazone), 5-{[4-((3,4-dihydro-6-hydroxy-2,5,7,8-tetramethyl-2H-1-benzopyran-2-yl)methoxy)-phenyl]-methyl}-thiazolidine-2,4-dione (troglitazone), 5-[6-(2-fluoro-benzyloxy)naphthalen-2-ylmethyl]-thiazolidine-2,4-dione (MCC555), 5-{[2-(2-naphthyl)-benzoxazol-5-yl]-methyl}thiazolidine-2,4-dione (T-174) and 5-(2,4-dioxothiazolidin-5-ylmethyl)-2-methoxy-N-(4-trifluoromethyl-benzyl)benzamide (KRP297).

Preferably, the antidiabetic thiazolidinedione is a compound of formula VIII,

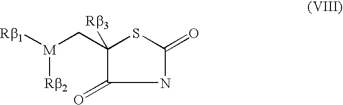

wherein
M represents
naphthyl, benzoxazolyl, dihydrobenzopyranyl, indole,
phenyl (optionally substituted by halogen) or phenylethynyl (optionally substituted by halogen);
   Rβ₁ represents halogen or a radical -QRβ₄, in which
Q can be oxygen, lower alkylen, carbonyl or —NH—,
Rβ₄ is
   naphthyl;
   phenyl, unsubstituted or substituted by 2,4-dioxo-5-thiazolidinyl; or
   lower alkyl or hydroxy lower alkyl, unsubstituted or substituted by
      a) indole or 2,3-dihydroindole,
      b) pyridyl, lower alkyl-pyridyl, N-lower alkyl-N-pyridylamino or halogenphenyl,
      c) dihydrobenzopyranyl, which is unsubstituted or substituted by hydroxy and lower alkyl,
      d) oxazolyl, which is substituted by lower alkyl and phenyl,
      e) cycloalkyl, which is unsubstituted or substituted by lower alkyl, or
      f) arylcycloalkylcarbonyl;
         Rβ₂ represents hydrogen or trifluoromethylphenyl-lower alkyl carbamoyl; and
         Rβ₃ represents hydrogen or arylsulfonyl;
         or a pharmaceutically acceptable salt thereof.

Preferably, the compound of formula VIII is selected from the group consisting of (S)-((3,4-dihydro-2-(phenyl-methyl)-2H-1-benzopyran-6-yl)methyl-thiazolidine-2,4-dione (englitazone), 5-{[4-(3-(5-methyl-2-phenyl-4-oxazolyl)-1-oxopropyl)-phenyl]-methyl}-thiazolidine-2,4-dione (darglitazone), 5-{[4-(1-methyl-cyclohexyl)methoxy)-phenyl]methyl}-thiazolidine-2,4-dione (ciglitazone), 5-{[4-(2-(1-indolyl)ethoxy)phenyl]methyl}-thiazolidine-2,4-dione (DRF2189), 5-{4-[2-(5-methyl-2-phenyl-4-oxazolyl)-ethoxy)]benzyl}-thiazolidine-2,4-dione (BM-13.1246), 5-(2-naphthylsulfonyl)-thiazolidine-2,4-dione (AY-31637), bis{4-[(2,4-dioxo-5-thiazolidinyl)methyl]phenyl}methane (YM268), 5-{4-[2-(5-methyl-2-phenyl-4-oxazolyl)-2-hydroxyethoxy]benzyl}-thiazolidine-2,4-dione (AD-5075), 5-[4-(1-phenyl-1-cyclopropanecarbonylamino)-benzyl]-thiazolidine-2,4-dione (DN-108) 5-{[4-(2-(2,3-dihydroindol-1-yl)ethoxy)phenyl]methyl}-thiazolidine-2,4-dione, 5-[3-(4-chloro-phenyl])-2-propynyl]-5-phenylsulfonyl)thiazolidine-2,4-dione, 5-[3-(4-chlorophenyl])-2-propynyl]-5-(4-fluorophenyl-sulfonyl)thiazolidine-2,4-dione, 5-[6-(2-fluoro-benzyloxy)naphthalen-2-ylmethyl]-thiazolidine-2,4-dione (MCC555), 5-{[2-(2-naphthyl)-benzoxazol-5-yl]-methyl}thiazolidine-2,4-dione (T-174) and 5-(2,4-dioxothiazolidin-5-ylmethyl)-2-methoxy-N-(4-trifluoromethyl-benzyl)benzamide (KRP297) or a pharmaceutically acceptable salt thereof.

More preferably, the compound of formula VIII is selected from the group consisting of 5-{[4-(2-(methyl-2-pyridinyl-amino)-ethoxy)phenyl]methyl}-thiazolidine-2,4-dione (rosiglitazone), 5-{[4-(2-(5-ethyl-2-pyridyl)ethoxy)phenyl]-methyl}thiazolidine-2,4-dione (pioglitazone) and 5-{[4-((3,4-dihydro-6-hydroxy-2,5,7,8-tetramethyl-2H-1-benzopyran-2-yl)methoxy)-phenyl]-methyl}-thiazolidine-2,4-dione (troglitazone), MCC555, T-174 and KRP297, especially rosiglitazone, pioglitazone and troglitazone, or a pharmaceutically acceptable salt thereof.

Other preferred antidiabetic thiazolidinedione under development are AZ242 (AstraZeneca) phase 2; KRP-297 (Kyorin, licensed to Merck) phase 1-2; MCC-555 (Mitsubishi Chemicals, licensed to J&J) phase 2; JTT-501 (Japan Tobacco, licensed to Pharmacia) phase 2.

The glitazones 5-{[4-(2-(5-ethyl-2-pyridyl)ethoxy)phenyl]-methyl}thiazolidine-2,4-dione (pioglitazone, EP 0 193 256 A1), 5-{[4-(2-(methyl-2-pyridinyl-amino)-ethoxy)phenyl]methyl}-thiazolidine-2,4-dione (rosiglitazone, EP 0 306 228 A1), 5-{[4-((3,4-dihydro-6-hydroxy-2,5,7,8-tetramethyl-2H-1-benzopyran-2-yl)methoxy)-phenyl]-methyl}thiazolidine-2,4-dione (troglitazone, EP 0 139 421), (S)-((3,4-dihydro-2-(phenyl-methyl)-2H-1-benzopyran-6-yl)methyl-thiazolidine-2,4-dione (englitazone, EP 0 207 605 B1), 5-(2,4-dioxothiazolidin-5-ylmethyl)-2-methoxy-N-(4-trifluoromethyl-benzyl)benzamide (KRP297, JP 10087641-A), 5-[6-(2-fluoro-benzyloxy)naphthalen-2-ylmethyl]thiazolidine-2,4-dione (MCC555, EP 0 604 983 B1), 5-{[4-(3-(5-methyl-2-phenyl-4-oxazolyl)-1-oxopropyl)-phenyl]-methyl}-thiazolidine-2,4-dione (darglitazone, EP 0 332 332), 5-(2-naphthylsulfonyl)-thiazolidine-2,4-dione (AY-31637, U.S. Pat. No. 4,997,948), 5-{[4-(1-methyl-cyclohexyl)methoxy)-phenyl]methyl}-thiazolidine-2,4-dione (ciglitazone, U.S. Pat. No. 4,287,200) are in each case generically and specifically disclosed in the documents cited in brackets beyond each substance, in each case in particular in the compound claims and the final products of the working examples, the subject-matter of the final products, the pharmaceutical preparations and the claims are hereby incorporated into the present application by reference to these publications. The preparation of DRF2189 and of 5-{[4-(2-(2,3-dihydroindol-1-yl)ethoxy)phenyl]methyl}-thiazolidine-2,4-dione is described in B. B. Lohray et al., J. Med. Chem. 1998, 41, 1619-1630; Examples 2d and 3 g on pages 1627 and 1628. The preparation of 5-[3-(4-chlorophenyl])-2-propynyl]-5-phenylsulfonyl)-thiazolidine-2,4-dione and the other compounds in which A is phenylethynyl mentioned herein can be carried out according to the methods described in J. Wrobel et al., J. Med. Chem. 1998, 41, 1084-1091.

In particular, MCC555 can be formulated as disclosed on page 49, lines 30 to 45, of EP 0 604 983 B1; englitazone as disclosed from page 6, line 52, to page 7, line 6, or analogous to Examples 27 or 28 on page 24 of EP 0 207 605 B1; and darglitazone and 5-{4-[2-(5-methyl-2-phenyl-4-oxazolyl)-ethoxy)]benzyl}-thiazolidine-2,4-dione (BM-13.1246) can be formulated as disclosed on page 8, line 42 to line 54 of EP 0 332 332 B1. AY-31637 can be administered as disclosed in column 4, lines 32 to 51 of U.S. Pat. No. 4,997,948 and rosiglitazone as disclosed on page 9, lines 32 to 40 of EP 0 306 228 A1, the latter preferably as its maleate salt. Rosiglitazone can be administered in the form as it is marketed e.g. under the trademark AVANDIA™. Troglitazone can be administered in the form as it is marketed e.g. under the trademarks ReZulin™, PRELAY™, ROMOZIN™ (in the United Kingdom) or NOSCAL™ (in Japan). Pioglitazone can be administered as disclosed in Example 2 of EP 0 193 256 A1, preferably in the form of the monohydrochloride salt. Corresponding to the needs of the single patient it can be possible to administer pioglitazone in the form as it is marketed e.g. under the trademark ACTOS™. Ciglitazone can, for example, be formulated as disclosed in Example 13 of U.S. Pat. No. 4,287,200.

For administration of a PPAR ANTIDIABETIC especially a glitazone to an adult diabetic patient (body weight: 50 kg), for instance, the dose per day is usually 0.01 to 1000 mg, preferably 0.1 to 500 mg. This dose can be administered once to several times a day. Especially, when pioglitazone hydrochloride is employed as the insulin sensitizer, the dose of pioglitazone hydrochloride per day is usually 7.5 to 60 mg, preferably 15 to 45 mg. When troglitazone is employed as the insulin sensitizer, the dose of troglitazone per day is usually 100 to 1000 mg, preferably 200 to 600 mg. When rosiglitazone (or its maleate) is employed as the insulin sensitizer, the dose of rosiglitazone per day is usually 1 to 12 mg, preferably 2 to 12 mg.

The glitazone is preferably pioglitazone, pioglitazone hydrochloride, troglitazone or rosiglitazone (or its maleate salt), especially preferably pioglitazone hydrochloride.

Pharmaceutical dosage forms containing combinations of antihyperglycemic drugs and thiazolidinedione derivatives have been proposed in the art. For example, EPO 0 749 751 teaches pharmaceutical compositions comprising an insulin sensitivity enhancer, which could be a thiazolidinedione compound, in combination with other antidiabetics. More specifically, EPO 0 749 751 teaches that the preferred insulin sensitivity enhancer is pioglitazone, which can be combined with other antidiabetics such as metformin, phenformin or buformin, and further that these drugs can be associated (mixed and/or coated) with conventional excipients to provide taste masking or sustained release. Another example of a combination of antihyperglycemic drugs and thiazolidinedione derivatives is U.S. Pat. No. 6,011,049. This patent teaches a single pharmaceutical composition that contains pioglitazone or troglitazone and metformin in slow release forms such as osmotic pumps or skin patches. Other combinations of antihyperglycemic drugs and thiazolidinedione derivatives can be found in U.S. Pat. Nos. 6,524,621; 6,475,521; 6,451,342 and 6,153,632 and PCT patent applications WO 01/3594 and WO 01/3594, which are incorporated herein by reference.

Also known in the art is WO 99/47125 and U.S. Pat. No. 6,099,862 that disclose a metformin osmotic tablet coated with an immediate release coating containing an antihyperglycemic or an hypoglycemic drug.

Although the prior art teaches pharmaceutical dosage formulations that contain both an antihyperglycemic compound and thiazolidinedione derivatives, the present invention provides numerous benefits over the prior art teachings as will be described below.

The dose of ACTOS® (pioglitazone) should not exceed 45 mg once daily in monotherapy or in combination with sulfonylurea, metformin, or insulin. ACTOS in combination with metformin may be initiated at 15 mg or 30 mg once daily. The current metformin dose can be continued upon initiation of ACTOS therapy. It is unlikely that the dose of metformin will require adjustment due to hypoglycemia during combination therapy with ACTOS. ACTOS is available in 15 mg, 30 mg, and 45 mg tablets

AVANDIA® (rosiglitazone) may be administered either at a starting dose of 4 mg as a single daily dose or divided and administered in the morning and evening. For patients who respond inadequately following 8 to 12 weeks of treatment, as determined by reduction in FPG, the dose may be increased to 8 mg daily as monotherapy or in combination with metformin. The dose of AVANDIA should not exceed 8 mg daily, as a single dose or divided twice daily. AVANDIA is available in 2 mg, 4 mg, and 8 mg tablets

Marketed combinations comprising metformin and a thiazolidinedione derivative can also be used according to the present invention. In particular it can be possible to administer rosiglitazone in combination with metformin in the form as it is marketed e.g. under the trademark AVANDAMET®. The dosage of antidiabetic therapy with AVANDAMET should be individualized on the basis of effectiveness and tolerability while not exceeding the maximum recommended daily dose of 8 mg/2,000 mg. AVANDAMET® provides different kind of tablets. Each tablet contains rosiglitazone as the maleate and metformin hydrochloride as follows: 1 mg/500 mg, 2 mg/500 mg, 4 mg/500 mg, 2 mg/1,000 mg, 4 mg/1,000 mg.

Non-glitazone type PPARγ agonists are especially N-(2-benzoylphenyl)-L-tyrosine analogues, e.g. GI-262570, and JTT501.

The term “dual PPARγ/PPARα agonists” as used herein means compounds which are at the same time PPARγ and PPARα agonists. Preferred dual PPARγ/PPARα agonists are especially those ω-[(oxoquinazolinylalkoxy)phenyl]alkanoates and analogs thereof, very especially the compound DRF-554158, described in WO 99/08501 and the compound NC-2100 described by Fukui in Diabetes 2000, 49(5), 759-767.

Dual acting PPAR alpha/gamma agonists particularly include those disclosed in co-owned international application PCT/EP02/13025 published on May 30, 2003 with publication No. WO 03/043985, particularly compounds of claim 19, most preferably compound 19 of Example 4, shown as compound 4-19, formula

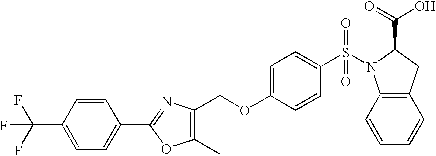

referred hereinafter as Compound L.

Another preferred PPAR alpha/gamma agonist is 3-isobutyl-8-(6-methoxy-isoquinolin-4-ylmethyl)-1-methyl-3,7-dihydro-purine-2,6-dione.

The preferred PPAR ANTIDIABETIC is selected from thiazolidinediones (glitazones) or dual PPARγ/PPARα agonists most preferably from thiazolidinediones.

The preferred PPAR ANTIDIABETIC is selected from Compound L, pioglitazone, pioglitazone hydrochloride, troglitazone or rosiglitazone or rosiglitazone maleate, or in each case the pharmaceutically acceptable salt of such a compound.

Multiple dosing regimens together, along with large doses, dose dependent absorption, poor bioavailability are not preferred since it leads to patient non-compliance, potential side effects & danger of overdosing. It is therefore imperative to shift from multiple dosing to a new and ideal once-a-day or twice-a-day dosing regimens. The applicant has therefore developed a novel patient-convenient, cost effective pharmaceutical dosage form to improve the quality of treatment.

Thus in a further aspect the present invention also relate to preferred pharmaceutical dosage form to provide the advantages already described herein and to improve the treatment of the diseases especially type 2 diabetes and IGT.

Preferably the combination is in the form of a fixed combination dosage form e.g. the three active ingredients are in the same table or capsule, as described below.
Dosage form wherein the DPP-IV inhibitor, the PPAR ANTIDIABETIC and metformin are in the form of a conventional immediate release formulation.
Dosage form wherein the 1) DPP-IV inhibitor and the PPAR ANTIDIABETIC are in the form of a conventional immediate release formulation and 2) metformin is in an extended release formulation.
Dosage form wherein 1) the PPAR ANTIDIABETIC is in the form of a conventional immediate release formulation; and 2) metformin and the DPP-IV inhibitor are in an extended release formulation.
Dosage form wherein the 1) DPP-IV inhibitor, and the PPAR ANTIDIABETIC are in the form of a conventional immediate release formulation and 2) metformin is partially in the conventional immediate release and partially in an extended release formulation.
Dosage form wherein 1) the PPAR ANTIDIABETIC is in the form of a conventional immediate release formulation and 2) metformin and the DPP-IV inhibitor are partially in the conventional immediate release formulation and partially in an extended release formulation.

The dosage form is preferably a solid pharmaceutical dosage form for oral administration.

The dosage form may be tablets or capsules. The tablet in the form of a multi-layered or bilayered tablet. The tablet may include a coating.

The capsules may include one or more of pellets, beads, granules, multiparticulates, tablets and powder.

The dosage form can comprise an extended release core of metformin and an immediate release layer of a PPAR ANTIDIABETIC and a DDP-IV inhibitor.

The dosage form can be a capsule comprising immediate release granules (or pellets or multiparticulates etc. as described below) of a PPAR ANTIDIABETIC and a DDP-IV inhibitor (in the same or in separate granules, pellets or multiparticulates) and extended release granules (or pellets or multiparticulates etc. as described below) of metformin.

The dosage form can be a tablet comprising an immediate release layer containing a PPAR ANTIDIABETIC and a DDP-IV inhibitor, and an extended release layer containing metformin. The dosage form can also be a tablet comprising an immediate release layer containing a PPAR ANTIDIABETIC a second immediate release layer containing a DDP-IV inhibitor and an extended release layer containing metformin.

The extended release layer may be a core and the immediate release layer may cover at least a portion of the core. The dosage form may be a multi-layered or bilayered dosage form. The core may be a matrix and the matrix may be a uniform mixture of metformin and one or more rate controlling polymers and may further include one or more pharmaceutically acceptable excipients. The immediate release outer layer may further include film-forming polymers and; optionally other pharmaceutically acceptable excipients. The film-forming polymers may be water-soluble polymers. The pharmaceutically acceptable excipients may be one or more of plasticizers, opacifiers and colorants.

After oral administration metformin may be released over a period of about 4 to about 36 hours and, more particularly, over a period of about 8 to about 24 hours.

Extended release formulations comprise pharmaceutically acceptable excipients (used in extended release layers or granules or tablets) and are well known in the art and are e.g. described in the herein cited prior art documents. Extended release layer may be a matrix and the matrix may have a uniform mixture of the metformin and one or more rate controlling polymers. The one or more rate-controlling polymers may be hydrophilic polymers, hydrophobic polymers, or a combination thereof. The matrix may further include one or more pharmaceutically acceptable excipients. The pharmaceutically acceptable excipients may be one or more I of diluents, lubricants, disintegrants, binders, glidants, coloring and flavoring agents. Preferably the formulation e.g. in the form of granules containing metformin is capable of being effectively compressed into a single tablet system exhibiting pH independent prolonged release of metformin.

Immediate release formulations comprise pharmaceutically acceptable excipients (used in extended release layers or granules or tablets) and are well known in the art and are e.g. described in the herein cited prior art documents. However an immediate release formulation can be limited only to the active ingredient or ingredients without the addition of a further pharmaceutically acceptable excipient.

The term ‘bilayered’ as used herein is meant to encompass solid dosage forms in which there are two separate drug layers, with only one surface in contact with each other. These may be prepared, for example, by compressing additional granulation on a previously compressed granulation or alternatively by feeding previously compressed tablets into a machine and compressing another granulation layer around the preformed tablets.

An example of a bi-layer tablet manufacturing method includes: (1) blending a quantity of a PPAR ANTIDIABETIC and DPP-IV inhibitor with various excipients, colorants, and/or other pharmaceutically acceptable excipients and additives to form an immediate release formulation, (2) blending a quantity of metformin with a rate-controlling polymer, and various excipients, colorants, and/or other pharmaceutical additives to form an extended release formulation, and (3) compressing a quantity of the immediate release formulation of the PPAR ANTIDIABETIC and DPP-IV inhibitor with a quantity of the extended release formulation of metformin to form a bi-layer tablet.

The manufacturing process can also involve the separate preparation of specially formulated granules containing Mefformin, the PPAR ANTIDIABETIC and the DPP-IV inhibitor and then compressing them (the three separate granules) into multilayered tablets exhibiting prolonged (preferably pH independent in-vitro) release of Mefformin and immediate release of the PPAR ANTIDIABETIC and DPP-IV inhibitor. Preferably pH independent in-vitro release of Mefformin up to a period of 8-12 hours

One of the embodiments includes providing a seal coat of hydrophilic polymers between the extended-release and immediate-release layers.

Other embodiments include modifications relating to coating the tablet with the polymer in order to modify the release of the drug. The solid dosage forms may be optionally coated with non-functional coatings well known in the art, or with coatings that further modify the release of the drug from the dosage form. All such modifications as may be done and understood by those who are skilled in the art are within the scope of the present invention. For example, one such modification includes making the composition into a layered tablet in which the composition provides extended release of more than one therapeutic agent, or extended release of one of the therapeutic agents and immediate or delayed release of the other therapeutic agent(s).

The invention also relates to the use of any one of the above described dosage form for the manufacture of a medicament for the prevention, delay of progression or treatment of conditions mediated by dipeptidylpeptidase-IV (DPP-IV), in particular diabetes, more particular type 2 diabetes mellitus, conditions of impaired glucose tolerance (IGT), conditions of impaired fasting plasma glucose, metabolic acidosis, ketosis, arthritis, obesity and osteoporosis.

The preparation of metformin (dimethyldiguanide) and its hydrochloride salt is state of the art and was disclosed first by Emil A. Werner and James Bell, J. Chem. Soc. 121, 1922, 1790-1794. Metformin, can be administered e.g. in the form as marketed under the trademarks GLUCOPHAGE™.

Comprised are likewise the corresponding stereoisomers as well as the corresponding polymorphs, e.g. crystal modifications, which are disclosed in the cited patent documents.

In a very preferred embodiment of the invention, the DPP-IV inhibitor is selected from (S)-1-[(3-hydroxy-1-adamantyl)amino]acetyl-2-cyano-pyrrolidine and (S)-1-{2-[5-cyanopyridin-2-yl)amino]ethyl-aminoacetyl}-2-cyano-pyrrolidine, the PPAR ANTIDIABETIC is selected from the group consisting of compound L, rosiglitazone, pioglitazone and the third compound is metformin, or in each case the pharmaceutically acceptable salt of such a compound.

In a second very preferred embodiment of the invention, the DPP-IV inhibitor is L-threo-isoleucyl thiazolidine (P32/98), MK-0431, 3-(aminomethyl)-2-isobuthyl-1-oxo-4-phenyl-1,2-dihydro-6-isoquinolinecarboxamide and 2-{[3-(aminomethyl)-2-isobuthyl-4-phenyl-1-oxo-1,2-dihydro-6-isoquinolyl]oxy}acetamide, and the PPAR ANTIDIABETIC is selected from the group consisting of compound L, rosiglitazone, pioglitazone and the third compound is metformin, or in each case the pharmaceutically acceptable salt of such a compound.

The term “prevention” means prophylactic administration of the combination to healthy patients to prevent the outbreak of the conditions mentioned herein. Moreover, the term “prevention” means prophylactic administration of such combination to patients being in a pre-stage of the conditions, especially diabetes, to be treated.

The term “delay of progression” used herein means administration of the combination, such as a combined preparation or pharmaceutical composition, to patients being in a pre-stage of the condition, especially diabetes, to be treated in which patients a pre-form of the corresponding condition is diagnosed.

The structure of the active agents identified by code nos., generic or trade names may be taken from the actual edition of the standard compendium “The Merck Index” or from databases, e.g. Patents International (e.g. IMS World Publications). The corresponding content thereof is hereby incorporated by reference. Any person skilled in the art is fully enabled to identify the active agents and, based on these references, likewise enabled to manufacture and test the pharmaceutical indications and properties in standard test models, both in vitro and in vivo.

The compounds to be combined can be present as pharmaceutically acceptable salts. If these compounds have, for example, at least one basic center, they can form acid addition salts. Corresponding acid addition salts can also be formed having, if desired, an additionally present basic center. The compounds having an acid group (for example COOH) can also form salts with bases. For example, the compounds to be combined can be present as a sodium salt, as a maleate or as a dihydrochloride. The active ingredient or a pharmaceutically acceptable salt thereof may also be used in form of a hydrate or include other solvents used for crystallization.

A combined preparation which comprises;

1) a dipeptidylpeptidase-IV (DPP-IV) inhibitor,
2) one antidiabetic selected from thiazolidinediones (glitazones), non-glitazone type PPARγ agonists, PPARα agonists or dual PPARγ/PPARα agonists and
3) metformin,
or in each case, the pharmaceutically acceptable salt of such a compound and optionally at least one, i.e., one or more, e.g. two, pharmaceutically acceptable carrier for simultaneous, separate or sequential use.

Especially covered is a “kit of parts” in the sense that the components,

1) a dipeptidylpeptidase-IV (DPP-IV) inhibitor,
2) one antidiabetic selected from thiazolidinediones (glitazones), non-glitazone type PPARγ agonists, PPARα agonists or dual PPARγ/PPARα agonists, and
3) metformin,
or in each case, the pharmaceutically acceptable salt of such a compound,
can be dosed independently or by use of different fixed combinations with distinguished amounts of the components, i.e. at different time points or simultaneously. The parts of the kit of parts can then, e.g., be administered simultaneously or chronologically staggered, that is at different time points and with equal or different time intervals for any part of the kit of parts. Preferably, the time intervals are chosen such that the effect on the treated disease or condition in the combined use of the parts is larger than the effect which would be obtained by use of only any one of the components. Preferably, there is at least one beneficial effect, e.g. a mutual enhancing of the effect of a DPP-IV inhibitor in combination with the antidiabetics 2) and 3) or in each case the pharmaceutically acceptable salt of such a compound, additional advantageous effects, less side effects, a combined therapeutical effect in a non-effective dosage of one or each of the components, and especially a potentiation or synergism, e.g. a more than additive effect, between a DPP-IV inhibitor in combination with the antidiabetics 2) and 3) or in each case the pharmaceutically acceptable salt of such a compound.

The nature of conditions mediated by DPP-IV, especially diabetes, conditions of impaired fasting plasma glucose, and IGT, is multifactorial. Under certain circumstances, drugs with different mechanisms of action may be combined. However, just considering any combination of drugs having different mode of action but acting in the similar field does not necessarily lead to combinations with advantageous effects.

All the more surprising is the experimental finding that the combined administration of

1) a DPP-IV inhibitor,
2) one antidiabetic selected from thiazolidinediones (glitazones), non-glitazone type PPARγ agonists, PPARα agonists or dual PPARγ/PPARα agonists, and
3) metformin,
results not only in a beneficial, especially a synergistic, therapeutic effect but also in additional benefits resulting from combined treatment such as a surprising prolongation of efficacy, a broader variety of therapeutic treatment and surprising beneficial effects on diseases and conditions associated with diabetes, e.g. less gain of weight.

Further benefits are that lower doses of the individual drugs to be combined according to the present invention can be used to reduce the dosage, for example, that the dosages need not only often be smaller but are also applied less frequently, or can be used in order to diminish the incidence of side effects. This is in accordance with the desires and requirements of the patients to be treated.

It can be shown by established test models and especially those test models described herein that the combination of 1) a DPP-IV inhibitor, especially (S)-1-{2-[5-cyanopyridin-2-yl)amino]ethyl-aminoacetyl}-2-cyano-pyrrolidine (DPP728) or (S)-1-[(3-hydroxy-1-adamantyl)amino]acetyl-2-cyano-pyrrolidine (LAF237), and 2) one anti-diabetic selected from thiazolidinediones (glitazones), non-glitazone type PPARγ agonists, PPARα agonists or dual PPARγ/PPARα agonists and 3) metformin results in a more effective (e.g. potentiation or synergistic effect) prevention or preferably treatment of conditions mediated by DPP-IV, in particular diabetes, especially type 2 diabetes mellitus, conditions of impaired fasting plasma glucose, and conditions of IGT.

The term “potentiation” shall mean an increase of a corresponding pharmacological activity or therapeutical effect, respectively. Potentiation of one component of the combination according to the present invention by co-administration of an other component according to the present invention means that an effect is being achieved that is greater than that achieved with one component alone or that is greater than the sum of effects of each component.

The term “synergistic” shall mean that the drugs, when taken together, produce a total joint effect that is greater than the sum of the effects of each drug when taken alone.

The person skilled in the pertinent art is fully enabled to select a relevant animal test model to prove the hereinbefore and hereinafter indicated therapeutic indications and beneficial effects. The pharmacological activity may, for example, be demonstrated following essentially an in-vivo test procedure in mice or in a clinical study as described hereinafter.

### In-Vivo Test in Mice for Blood Glucose Control

ICR-CDI mice (male, five weeks old, body weight: about 20 g) are abstained from food for 18 hours, and then used as test subjects. The combination according to the present invention and the active ingredients alone are suspended in 0.5% CMC-0.14M sodium chloride buffer solution (pH 7.4). The solution thus obtained is administered orally in fixed volume amounts to the test subjects. After predetermined time, the percentage decrease of the blood glucose against the control group is determined.

Clinical Double-Blind, Randomized, Parallel-Group Study in Subjects with Type 2 Diabetes Mellitus Inadequately Controlled on Diet Alone

This study proves in particular the synergism of the claimed combined preparation or pharmaceutical composition, respectively. The beneficial effects on conditions mediated by DPP-IV, in particular type 2 diabetes mellitus can be determined directly through the results of this study or by changes in the study design which are known as such to a person skilled in the art.

The study is, in particular, suitable to compare the effects of monotherapy with COMBINATION PARTNERS OF THE INVENTION with those of a combination of DPP-IV inhibitor plus one of these compounds on glycemic control.

The term “COMBINATION PARTNERS OF THE INVENTION” means 1) an antidiabetic selected from thiazolidinediones (glitazones), non-glitazone type PPARγ agonists, PPARα agonists or dual PPARγ/PPARα agonists, and 2) metformin.

Subjects with a diagnosis of type 2 diabetes mellitus who have not achieved near normoglycemia (HbA_{1c} <6.8%) on diet only are chosen for this trial. The effects on glycemic control achieved with DPP-IV monotherapy, monotherapy with one antidiabetic selected from thiazolidinediones (glitazones), non-glitazone type PPARγ agonists, PPARα agonists or dual PPARγ/PPARα agonists, or monotherapy with metformin, and the combination therapy of 1) a DPP-IV inhibitor, plus 2) one antidiabetic selected from thiazolidinediones (glitazones), non-glitazone type PPARγ agonists, PPARα agonists or dual PPARγ/PPARα agonists, and 3) metformin, are determined in this study after 24 weeks with the control achieved on placebo, all subjects continuing with the same diet as in the period before treatment. Measures of glycemic control are validated surrogate endpoints for the treatment of diabetes. HbA_{1c} is the single most reliable measurement for assessing glycemic control (D. Goldstein et al, Tests of Glycemia in Diabetes; Diabetes Care 1995, 18(6), 896-909) and is the primary response variable in this study. Since glycosylation of hemoglobin is determined by the glucose concentration at the time each red blood cell is made, HbA_{1c} provides an estimate of mean blood glucose for the previous three months.

Before starting with the double-blind treatment for 24 weeks, the subjects are administered for four weeks the placebos matching with the 1) DPP-IV inhibitor, e.g. DPP728 and LAF237, before breakfast, lunch and dinner, and the placebos matching with, and 2) one antidiabetic selected from thiazolidinediones (glitazones), non-glitazone type PPARγ agonists, PPARα agonists or dual PPARγ/PPARα agonists, and 3) metformin (period I). For example, if the antidiabetic thiazolidinedione pioglitazone is chosen for the study, the placebos matching with pioglitazone are preferably administered in period I with breakfast only. If metformin is chosen for the study, matching placebos are preferably administered before breakfast and dinner.

The subjects are then separated into four treatment groups for the 24-week double-blind study (period II) as depicted in Tables 1 to 11 (including the alternative combination options) for the case that DPP728 is chosen as the DPP-IV inhibitor and the antidiabetic thiazolidinedione pioglitazone or rosiglitazone, and metformin are chosen as the combination partner.

### Examples for Combinations to be Administered and Treatment Regimens

If the three active ingredients have to be administered once a day, a dosage form as described above would be a preferred embodiment.

Preferred treatment regimen according to the invention are described below. In the below examples, rosiglitazone is in form of the rosiglitazone maleate.

TABLE 1
LAF237 plus rosiglitazone plus metformin

LAF237 50 mg* or 100 mg* + rosiglitazone placebo** + metformin
placebo***
rosiglitazone 2 mg** + LAF237 placebo* + metformin placebo***
Metformin 500 mg*** + LAF237 placebo* + rosiglitazone placebo**
LAF237 50 mg* or 100 mg* + rosiglitazone 2 mg** + Metformin
500 mg***
LAF237 placebo* + rosiglitazone placebo** + metformin placebo***
*administered once or twice daily i.e. 50 mg or 100 mg or 2 × 50 mg
**administered once daily preferably concomitantly with LAF237
***administered once daily if in the form of extended-release tablets preferably with the evening meal or with LAF and rosiglitazone; or twice a day (250 mg × 2) if not in the form of extended-release tablets preferably with meals.

TABLE 2
LAF237 plus rosiglitazone plus metformin

LAF237 50 mg* or 100 mg* + rosiglitazone placebo** + metformin
placebo***
rosiglitazone 2 mg** + LAF237 placebo* + metformin placebo***
Metformin 500 mg*** + LAF237 placebo* + rosiglitazone placebo**
LAF237 50 mg* or 100 mg* + rosiglitazone 2 mg** + Metformin
500 mg***
LAF237 placebo* + rosiglitazone placebo** + metformin placebo***
*administered once daily or twice daily i.e. 50 mg or 100 mg or 2 × 50 mg
**administered once daily preferably concomitantly with LAF237
***administered once daily if in the form of extended-release tablets preferably with the evening meal or with LAF and rosiglitazone; or twice a day (250 mg × 2) if not in the form of extended-release tablets preferably with meals.

TABLE 3
LAF237 plus rosiglitazone plus metformin

LAF237 50 mg* or 100 mg* + rosiglitazone placebo** + metformin
placebo***
rosiglitazone 2 mg** + LAF237 placebo* + metformin placebo***
Metformin 850 mg*** + LAF237 placebo* + rosiglitazone placebo**
LAF237 50 mg* or 100 mg* + rosiglitazone 2 mg** + Metformin
850 mg***
LAF237 placebo* + rosiglitazone placebo** + metformin placebo***
*administered once daily or twice daily i.e. 50 mg or 100 mg or 2 × 50 mg
**administered once daily e.g. with the breakfast or divided and administered twice daily e.g. the morning and in the evening, preferably concomitantly with LAF237
***administered once daily with meal e.g. breakfast or with LAF and rosiglitazone.

TABLE 4
LAF237 plus rosiglitazone plus metformin

LAF237 50 mg* or 100 mg* + rosiglitazone placebo** + metformin
placebo***
rosiglitazone 4 mg** + LAF237 placebo* + metformin placebo***
Metformin 850 mg*** + LAF237 placebo* + rosiglitazone placebo**
LAF237 50 mg* or 100 mg* + rosiglitazone 4 mg** + Metformin
850 mg***
LAF237 placebo* + rosiglitazone placebo** + metformin placebo***
*administered once daily e.g. with the breakfast or twice daily i.e. 50 mg or 100 mg or 2 × 50 mg
**administered once daily e.g. with the breakfast or divided and administered twice daily i.e. 2 × 2 mg e.g. the morning and in the evening, preferably concomitantly with LAF237
***administered once daily with meal e.g. breakfast or with LAF and rosiglitazone.

TABLE 5
LAF237 plus rosiglitazone plus metformin

LAF237 50 mg* or 100 mg* + rosiglitazone placebo** + metformin
placebo***
rosiglitazone 2 mg** + LAF237 placebo* + metformin placebo***
Metformin 1000 mg*** + LAF237 placebo* + rosiglitazone placebo**
LAF237 50 mg* or 100 mg* + rosiglitazone 2 mg** + Metformin
1000 mg***
LAF237 placebo* + rosiglitazone placebo** + metformin placebo***
*administered once daily e.g. with the breakfast or twice daily i.e. 50 mg or 100 mg or 2 × 50 mg
**administered once daily preferably concomitantly with LAF237
***administered twice a day with meals (2 × 500 mg) or once daily if in the form of extended-release tablets preferably with the evening meal or with LAF and rosiglitazone.

TABLE 6
LAF237 plus rosiglitazone plus metformin

LAF237 50 mg* or 100 mg* + rosiglitazone placebo** + metformin
placebo
rosiglitazone 4 mg** + LAF237 placebo* + metformin placebo
Metformin 1000 mg + LAF237 placebo* + rosiglitazone placebo**
LAF237 50 mg* or 100 mg* + rosiglitazone 4 mg** + Metformin
1000 mg
LAF237 placebo* + rosiglitazone placebo** + metformin placebo
*administered once daily e.g. with the breakfast or twice daily i.e. 50 mg or 100 mg or 2 × 50 mg
**administered once daily e.g. with the breakfast or divided and administered twice daily i.e. 2 × 2 mg e.g. the morning and in the evening, preferably concomitantly with LAF237
***administered twice a day with meals (2 × 500 mg) or once daily if in the form of extended-release tablets preferably with the evening meal or with LAF and rosiglitazone.

TABLE 7
LAF237 plus pioglitazone plus metformin

LAF237 50 mg* or 100 mg* + pioglitazone placebo** + metformin
placebo***
pioglitazone 15 mg** + LAF237 placebo* + metformin placebo***
Metformin 500 mg*** + LAF237 placebo*+ pioglitazone placebo**
LAF237 50 mg* or 100 mg* + pioglitazone 15 mg** + metformin
500 mg***
LAF237 placebo* + pioglitazone placebo** + metformin placebo***
*administered once daily e.g. with the breakfast or twice daily i.e. 50 mg or 100 mg or 2 × 50 mg
**administered once daily, preferably concomitantly with LAF237
***administered once daily if in the form of extended-release tablets preferably with the evening meal or with LAF and pioglitazone; or twice a day (250 mg × 2) if not in the form of extended-release tablets preferably with meals.

TABLE 8
LAF237 plus pioglitazone plus metformin

LAF237 50 mg* or 100 mg* + pioglitazone placebo** + metformin
placebo***
pioglitazone 15 mg** + LAF237 placebo* + metformin placebo***
Metformin 850 mg*** + LAF237 placebo* + pioglitazone placebo**
LAF237 50 mg* or 100 mg* + pioglitazone 15 mg** + metformin
500 mg***
LAF237 placebo* + pioglitazone placebo** + metformin placebo***
*administered once daily e.g. with breakfast or twice daily i.e. 50 mg or 100 mg or 2 × 50 mg
**administered once daily preferably once daily concomitantly with LAF237
***administered once daily with meal e.g. breakfast or with LAF and pioglitazone.

TABLE 9
LAF237 plus pioglitazone plus metformin

LAF237 50 mg* or 100 mg* + pioglitazone placebo** + metformin
placebo***
pioglitazone 30 mg** + LAF237 placebo* + metformin placebo***
Metformin 500 mg*** + LAF237 placebo* + pioglitazone placebo*
LAF237 50 mg* or 100 mg* + pioglitazone 30 mg** + metformin
500 mg***
LAF237 placebo* + pioglitazone placebo** + metformin placebo***
*administered once daily e.g. with breakfast or twice daily i.e. 50 mg or 100 mg or 2 × 50 mg
**administered once daily preferably once daily concomitantly with LAF237
***administered once daily if in the form of extended-release tablets preferably with the evening meal or with LAF and pioglitazone; or twice a day (250 mg × 2) if not in the form of extended-release tablets preferably with meals.

TABLE 10
LAF237 plus pioglitazone plus metformin

LAF237 50 mg* or 100 mg* + pioglitazone placebo** + metformin
placebo***
pioglitazone 15 mg** + LAF237 placebo* + metformin placebo***
Metformin 1000 mg*** + LAF237 placebo* + pioglitazone placebo**
LAF237 50 mg* or 100 mg* + pioglitazone 15 mg** + metformin
1000 mg***
LAF237 placebo* + pioglitazone placebo** + metformin placebo***
*administered once daily e.g. with breakfast or twice daily i.e. 50 mg or 100 mg or 2 × 50 mg
**administered once daily preferably once daily concomitantly with LAF237
***administered twice a day with meals (2 × 500 mg) or once daily if in the form of extended-release tablets preferably with the evening meal or with LAF and pioglitazone.

TABLE 11
LAF237 plus pioglitazone plus metformin

LAF237 50 mg* or 100 mg* + pioglitazone placebo** + metformin
placebo***
pioglitazone 45 mg** + LAF237 placebo* + metformin placebo***
Metformin 500 mg*** + LAF237 placebo* + pioglitazone placebo*
LAF237 50 mg* or 100 mg* + pioglitazone 45 mg** + metformin
500 mg***
LAF237 placebo* + pioglitazone placebo** + metformin placebo***
*administered once daily e.g. with breakfast or twice daily i.e. 50 mg or 100 mg or 2 × 50 mg
**administered once daily preferably once daily concomitantly with LAF237
***administered once daily if in the form of extended-release tablets preferably with the evening meal or with LAF and pioglitazone; or twice a day (250 mg × 2) if not in the form of extended-release tablets preferably with meals.

LAF tablets contain either 50 mg of the compound or matching placebo. Pioglitazone or Rosiglitazone tablets, and metformin tablets can be purchased commercially and overencapsulated to match the corresponding placebo capsules.

A daily dosage 100 mg of LAF237 or a pharmaceutical salt thereof is administered, once a day or divided in two separate administrations of 50 mg preferably with or before separate meals.

Alternatively, the invention also relates to the above described combination therapy wherein a daily dosage of 8 mg of rosiglitazone or rosiglitazone maletate or any other salt is administered once daily or divided twice daily i.e. 2×4 mg

Alternatively, the invention also relates to the above described combination therapy wherein 2550 mg of metformin are administered in 3 times a day i.e. 3×850 mg, or 2000 mg of metformin is administered twice daily i.e. 2×1000 mg, or 1500 mg are administered twice a day i.e. 2×750 or 3 times a day i.e. 3×500 mg.

A combination according to the invention, wherein the dosage of the 3 active ingredients is as depicted in the above tables 1 to 11 (including the 2550 mg, 2000 mg or 1500 mg of metformin administration alternative mentioned above).

LAF237 is preferably administered with or before the meal e.g. 30 minutes before the meal.

The subjects are then separated into four treatment groups for the 24-week double-blind study (period II) as depicted in Table 1. Approximately 170 subjects are randomized per treatment group. The total study duration including the run-in period for each subject is 28 weeks. Statistical analysis can be carried out by methods known in the art.

The subject is advised not to take the morning dose of study medication or eat breakfast on the day of a scheduled study visit. The morning dose is administered by site personnel after the collection of all fasting laboratory samples and completion of all study procedures. Visits are scheduled to be performed at 2 week intervals during period I, and 4 to 8 week intervals during period II. Subjects have fasted for at least 7 hours at the time of each visit. All blood samples for laboratory evaluations are drawn between 7:00 AM and 10:00 AM. All tests are conducted in accordance with Good Laboratory Practice principles following procedures known in the art.

HbA_{1c} is measured by High Performance Liquid Chromatography (HPLC) using the ion-exchange method on a Bio-Rad Diamat analyzer. A back-up affinity method are used if hemoglobin variants or hemoglobin degradation peaks are observed.

Further parameters to be determined are fasting plasma glucose (FPG), fasting lipids (total, HDL (high density lipoprotein)- and LDL (low density lipoprotein)-cholesterol, and triglycerides) and body weight. FPG will be measured using the hexokinase method and LDL-cholesterol will be calculated using the Friedewald formula if triglycerides are <400 mg/dL (4.5 mmol/l).

Various parameters of the study described above can be modified, e.g. in order to optimize the dosage for special diseases or indications mentioned herein, to cope with tolerability problems during the study or to obtain similar or identical results with less efforts. For example, a different subject population can be involved in such a clinical trial, e.g. subjects with a diagnosis of type 2 diabetes mellitus who have achieved near normoglycemia (HbA_{1c} <6.8%) on diet alone, subjects with diseases other than diabetes mellitus, e.g. other metabolic disorders, or subjects selected by other criteria, such as age or sex; the subject number can be decreased, e.g. to a number of between 70 and 150, especially 100 or 120, subjects per treatment group; treatment groups (listed exemplary in Table 1) can be deleted, i.e. for example to carry out a study with a comparison of the combination of a DPP-IV inhibitor and COMBINATION PARTNERS OF THE INVENTION versus a DPP-IV inhibitor alone or metformin alone or pioglitazone alone or rosiglitazone alone or a dual combination i.e. COMBINATION PARTNERS OF THE INVENTION (e.g. metformin+pioglitazone) alone; the term of the placebo run-in period (period I) can be changed, i.e. it can be extended, shortened or deleted; the visit schedule can be extended, e.g. to every 10, 12 or 14 weeks; the visit instructions can be changed, e.g. the instruction that blood samples for laboratory evaluations have to be drawn between 7:00 AM and 10:00 AM; HbA_{1c} can be determined by other means; or one or more of the parameters to be determined during the study mentioned above, e.g. FPG or fasting lipids, can be deleted or the determination of additional parameters (see below) can be added.

Additional parameters can be determined in the course of the study, e.g. by additional tests. Such additional tests can comprise the analysis of body liquids in order to determine amounts or numbers for parameters such as those listed below and can serve e.g. the purpose of determining the tolerability of the administered active ingredients: determination of hematocrit and hemoglobin, platelet count, erythrocyte count, total and differential leukocyte count (basophils, eosinophils, lymphocytes, monocytes, segmented neutrophils and total neutrophils); determination of albumin, alkaline phosphatase, alanine amino transferase (serum glutamic pyruvic transaminase), aspartate amino transferase (serum glutamic oxalacetic transaminase), blood urea nitrogen or urea, bicarbonate, calcium, chloride, total creatine phosphokinase (CPK), creatine phosphokinase muscle-brain fraction isoenzyme (if CPK is elevated), direct bilirubin, creatinine, γ-glutamyl transferase, lactate dehydrogenase, potassium, sodium, total bilirubin, total protein and uric acid in the blood; determination of bilirubin, glucose, ketones, pH, protein, and specific gravity in the subjects urine; determination of body weight, blood pressure (systolic and diastolic, after 3 minutes sitting) and radial pulse (after 3 minutes sitting).

The results of the studies show that the combination according to the present invention can be used for the prevention and preferably the treatment of conditions mediated by DPP-IV, in particular type 2 diabetes mellitus. The combination of the present invention can also be used for the prevention and preferably the treatment of other condition mediated by DPP-IV.

Furthermore, in a number of combinations as disclosed herein the side-effects observed with one of the components surprisingly do not accumulate on application of the combination.

Preferably, the jointly therapeutically effective amounts of a DPP-IV inhibitor in free or pharmaceutically acceptable salt form and at least one further pharmaceutically active compound are administered simultaneously or sequentially in any order, separately or in a fixed combination.

The condition mediated by DPP-IV is preferably selected from the group consisting of diabetes, impaired fasting plasma glucose, impaired glucose tolerance, metabolic acidosis, ketosis, arthritis, obesity and osteoporosis.

Very preferably, the condition mediated by DPP-IV is type 2 diabetes mellitus.

It is one objective of this invention to provide a pharmaceutical composition comprising a quantity, which is jointly therapeutically effective against conditions mediated by DPP-IV, in particular diabetes, more especially type 2 diabetes mellitus, conditions of impaired fasting plasma glucose, and conditions of IGT, of a DPP-IV inhibitor (i) or a pharmaceutically acceptable salt thereof and (ii) COMBINATIONS PARTNER OF THE INVENTION and at least one pharmaceutically acceptable carrier.

The pharmaceutical compositions according to the invention can be prepared in a manner known per se and are those suitable for enteral, such as oral or rectal, and parenteral administration to mammals (warm-blooded animals), including man, comprising a therapeutically effective amount of the pharmacologically active compound, alone or in combination with one or more pharmaceutically acceptable carries, especially suitable for enteral or parenteral application.

The novel pharmaceutical preparations contain, for example, from about 10% to about 100%, e.g., 80% or 90%, preferably from about 20% to about 60%, of the active ingredient. Pharmaceutical preparations according to the invention for enteral or parenteral administration are, for example, those in unit dose forms, such as sugar-coated tablets, tablets, capsules or suppositories, and furthermore ampoules. These are prepared in a manner known per se, for example by means of conventional mixing, granulating, sugar-coating, dissolving or lyophilizing processes. Thus, pharmaceutical preparations for oral use can be obtained by combining the active ingredient with solid carriers, if desired granulating a mixture obtained, and processing the mixture or granules, if desired or necessary, after addition of suitable excipients to give tablets or sugar-coated tablet cores.

In this composition, components (i) and (ii) can be administered together, one after the other or separately in one combined unit dose form or in two or three separate unit dose forms (e.g. a DPP-IV inhibitor+metformin in one unit dose form and a PPAR ANTIDIABETIC in a separate unit dose form, or a DPP-IV inhibitor+a PPAR ANTIDIABETIC in one unit dose form and metformin in a separate unit dose form or each compound in a separate unit dose form). In one preferred embodiment of the invention, the unit dose form is a fixed combination. In a fixed combination the components e.g. a DPP-IV inhibitor+metformin or a DPP-IV inhibitor+a PPAR ANTI DIABETIC or metformin+a PPAR ANTI DIABETIC or preferably a DPP-IV inhibitor+metformin+a PPAR ANTIDIABETIC are administered in the form of a single galenic formulation, e.g. a single tablet or a single infusion.

A further aspect of the present invention is the use of a pharmaceutical composition comprising a DPP-IV inhibitor and at least one further COMBINATION PARTNER OF THE INVENTION, in each case in free form or in form of a pharmaceutically acceptable salt thereof for the preparation of a pharmaceutical preparation for the prevention or treatment of conditions mediated by DPP-IV, in particular diabetes, more especially type 2 diabetes mellitus, conditions of impaired fasting plasma glucose, and conditions of IGT.

A therapeutically effective amount of each of the components of the COMBINATION OF THE INVENTION may be administered simultaneously or sequentially and in any order, and the components may be administered separately or as a fixed combination. For example, the method of treatment of the invention may comprise (i) administration of a DPP-IV inhibitor in free or pharmaceutically acceptable salt form and (ii) administration of the COMBINATION PARTNERS OF THE INVENTION, simultaneously or sequentially in any order, in jointly therapeutically effective amounts, preferably in synergistically effective amounts, e.g. in daily dosages corresponding to the ratios described herein.

The corresponding active ingredient or a pharmaceutically acceptable salt thereof may also be used in form of a hydrate or include other solvents used for crystallization.

Furthermore, the term administering also encompasses the use of prodrugs of any of the anti-diabetic drugs that convert in vivo to the selective anti-diabetic drug. The instant invention is therefore to be understood as embracing all such regimes of simultaneous or alternating treatment and the term “administering” is to be interpreted accordingly.

The invention relates in particular to a commercial package comprising jointly therapeutically effective amounts of a DPP-IV inhibitor, in free or pharmaceutically acceptable salt form, and the COMBINATION PARTNERS OF THE INVENTION together with instructions for use thereof in the treatment of conditions mediated by DPP-IV, in particular diabetes, more especially type 2 diabetes mellitus, conditions of impaired fasting plasma glucose, and conditions of IGT.

### Use+Methode

In a further aspect of the present invention relate to the use of a dipeptidylpeptidase-IV (DPP-IV) inhibitor, in combination with i) one antidiabetic selected from thiazolidinediones (glitazones), non-glitazone type PPARγ agonists, PPARα agonists or dual PPARγ/PPARα agonists and with ii) metformin, or in each case the pharmaceutically acceptable salt of such a compound, for the manufacture of a medicament for the prevention, delay of progression or treatment of conditions mediated by dipeptidylpeptidase-IV (DPP-IV), in particular diabetes, more particular type 2 diabetes mellitus, conditions of impaired glucose tolerance (IGT), conditions of impaired fasting plasma glucose, metabolic acidosis, ketosis, arthritis, obesity and osteoporosis.

Use of LAF237 or a pharmaceutical salt thereof, in combination with;
i) metformin or a salt thereof, and
ii) pioglitazone or rosiglitazone or in any case pharmaceutical salts thereof,
   for the manufacture of a medicament for the prevention, delay of progression or treatment of conditions mediated by dipeptidylpeptidase-IV (DPP-IV), in particular diabetes, more particular type 2 diabetes mellitus, conditions of impaired glucose tolerance (IGT), conditions of impaired fasting plasma glucose, metabolic acidosis, ketosis, arthritis, obesity and osteoporosis wherein the above described treatment schedule are used tables 1 to 11 (including the 2550 mg, 2000 mg or 1500 mg of metformin administration alternative mentioned above).

A further aspect of the present invention is a method of treating a condition mediated by DPP-IV, in particular type 2 diabetes mellitus, comprising administering to a warm-blooded animal in need thereof jointly therapeutically effective amounts of a DPP-IV inhibitor in free or pharmaceutically acceptable salt form, and the COMBINATION PARTNERS OF THE INVENTION. Preferably, in this method of treating the active ingredients are administered simultaneously or sequentially in any order, separately or in a fixed combination. In one preferred embodiment of such method the jointly therapeutically effective amounts of a dipeptidylpeptidase-IV inhibitor in free or pharmaceutically acceptable salt form and the COMBINATION PARTNERS OF THE INVENTION are provided as a combined preparation.

A method of treating a condition mediated by DPP-IV, in particular type 2 diabetes mellitus, comprising administering to a warm-blooded animal in need thereof jointly therapeutically effective amounts of LAF237 or a pharmaceutical salt thereof, in combination with;
i) metformin or a salt thereof, and
ii) pioglitazone or rosiglitazone or in any case pharmaceutical salts thereof,

Method or use as described above, wherein a daily dosage of 50 mg or 100 mg of LAF237 or a pharmaceutical salt thereof is administered.

Method or use as described above, wherein a daily dosage 100 mg of LAF237 or a pharmaceutical salt thereof is administered, and divided in two separate administrations of 50 mg, preferably with or before separate meals.

Method or use as described above, wherein a daily dosage of 15, 30 or 45 mg of pioglitazone or a pharmaceutical salt thereof is administered.

Method or use as described above, wherein a daily dosage of 4, or 8 mg of rosiglitazone or rosiglitazone maleate or any pharmaceutical salt thereof is administered. Method or use as described above, wherein a daily dosage of 4 or 8 mg of rosiglitazone or rosiglitazone maleate or any pharmaceutical salt thereof is administered as a single dose or divided twice daily e.g. 2×2 mg or 2×4 mg.

Method or use as described above, wherein a daily dosage of 250, 500, 750, 850, 1000, 1500, 2000 or 2550 mg of metformin or a pharmaceutical salt thereof is administered.

Method or use as described herein, wherein at least one tablet is administered in the form of a fixed combination comprising;

i) LAF237 and metformin or in any case a salt thereof,
ii) pioglitazone and metformin or in any case a salt thereof,
iii) rosiglitazone and metformin or in any case a salt thereof,
iv) LAF237 and pioglitazone or in any case a salt thereof,
v) LAF237 and rosiglitazone or in any case a salt thereof,
vi) LAF237 and pioglitazone and metformin or in any case a salt thereof, or
vii) LAF237 and rosiglitazone and metformin or in any case a salt thereof.

Furthermore, the present invention provides a method of treating conditions of impaired glucose tolerance and impaired fasting plasma glucose comprising administering to a warm-blooded animal in need thereof jointly therapeutically effective amounts of a DPP-IV inhibitor in free or pharmaceutically acceptable salt form, and the COMBINATION PARTNERS OF THE INVENTION.

Furthermore, the invention relates to a method of improving the bodily appearance of a mammal which comprises orally administering to said mammal, including man, especially man suffering from a metabolic disorder, in particular type 2 diabetes, a combined preparation or pharmaceutical composition described herein in a dosage effective to influence, e.g., to increase or decrease, the glucose metabolism, or to influence the body weight by other mechanisms, and repeating said dosage until a cosmetically beneficial loss of body weight has occurred. Such combinations described herein can also be used to prevent, for cosmetic reasons, a further increase in body weight in humans experiencing such an increase. Moreover, the invention relates to the combinations described herein useful for improving the bodily appearance of a mammal, especially a human being, and the use of such combinations in order to improve the bodily appearance of a mammal, especially a human being. Overweight is one of the risk factors for developing a metabolic disorder, in particular type 2 diabetes, and at the same time often the result of such a metabolic disorder, especially type 2 diabetes. Furthermore, a number of antidiabetics are known to cause weight gain. Hence, humans suffering from metabolic disorders, especially type 2 diabetes, are often faced with overweight. Therefore, the cosmetically beneficial loss of body weight can be effected especially in humans suffering from a metabolic disorder, such as type 2 diabetes. The combinations described herein can also be used to replace or complement an antidiabetic drug taken by a human suffering from type 2 diabetes in order to prevent, for cosmetic reasons, a further increase of the body weight.

The dosage range of the combination of a DPP-IV inhibitor and the COMBINATION PARTNERS OF THE INVENTION to be employed depends upon factors known to the person skilled in the art including species of the warm-blooded animal, body weight and age, the nature and severity of the condition to be treated, the mode of administration and the particular substance to be employed. Unless stated otherwise herein, the DPP-IV inhibitor and the COMBINATION PARTNERS OF THE INVENTION are preferably divided and administered from one to four times per day.

The weight ratio of the daily doses of DPP728 or LAF237 or a pharmaceutically acceptable salt thereof to the COMBINATION PARTNERS OF THE INVENTION may vary within wide limits depending in particular on the needs of the warm-blooded animal treated.

In a more preferred embodiment of the invention the following weight ratios of DPP728 or LAF237 or a pharmaceutically acceptable salt thereof to the COMBINATION PARTNERS OF THE INVENTION should be administered in order to obtain a synergistic effect of the components:

| further pharmaceutically | DPP728 or LAF237/further pharmaceutically |
|---|---|
| active compound | active compound |
| Troglitazone | between 1:1 and 1:10, |
| | preferably between 1:2 and 1:6, e.g. 1:4 |
| Metformin | between 4:1 and 1:60, |
| | preferably between 1:1 and 1:10, e.g. 1:6 |

If the warm-blooded animal is a human of about 70 kg body weight the dosages of the at least one further pharmaceutically active compounds are preferably the following:

| pharmaceutically active | | |
|---|---|---|
| compound | preferred dosage | most preferred dosage |
| ciglitazone | about 0.25 to 200 mg/kg body | about 0.5 to 50 mg/kg body |
| | weight of the patient per day | weight of the patient per day |
| darglitazone | about 0.05 to 50 mg/kg body | about 0.05 to 5 mg/kg body |
| | weight of the patient per day | weight of the patient per day |
| DN-108 | about 0.25 to 200 mg/kg body | about 5 to 100 mg/kg body |
| | weight of the patient per day | weight of the patient per day |
| DPP728 | about 25 to 1000 mg/day | about 150 to 300 mg/day |
| englitazone | about 0.05 to 50 mg/kg body | about 0.05 to 5 mg/kg body |
| | weight | weight |
| KRP297 | about 0.1 to 2500 mg/day | about 1 to 1000 mg/day |
| MCC555 | about 0.1 to 2000 mg/day | about 0.5 to 100 mg/day |
| metformin | about 250 to 1500 mg/day | about 500 to 1250, e.g. 1000, |
| | | mg/day |
| pioglitazone | about 0.1 to 1000 mg/day | about 10 to 150, for example |
| | | 15, 30, 45 or 90, mg/day |
| rosiglitazone | about 0.1 to 500 mg/day | about 1 to 20, for example 1, |
| | | 2, 4 or 8, mg/day |
| troglitazone | about 0.1 to 2000 mg/day | about 50 to 1000 for example |
| | | 100, 200, 400, 600 or 800, |
| | | mg/day, mg/day |
| 5-[3-(4-chlorophenyl])-2- | about 0.1 to 2500 mg/day | about 1 to 1000 mg/day |
| propynyl]-5-phenylsulfonyl)- | | |
| thiazolidine-2,4-dione | | |
| 5-[3-(4-chlorophenyl])-2- | about 0.1 to 2500 mg/day | about 1 to 1000 mg/day |
| propynyl]-5-(4-fluoro- | | |
| phenylsulfonyl)thiazolidine- | | |
| 2,4-dione | | |
| N-(N′-substituted glycyl)-2- | about 0.1 to 250 mg/kg body | about 1 to 100 mg/kg body |
| cyanopyrrolidine of formula I | weight of the patient per day | weight of the patient per day |

The following Examples shall illustrate the invention described above; they are not, however, intended to limit the scope of the invention in any way.

Combination, combined preparation, pharmaceutical preparation or formulation, dosage form, method of treatment, use, Kit of Parts or commercial kit according to the present invention wherein;
the preferred PPAR ANTIDIABETIC is selected from Compound L, pioglitazone, pioglitazone hydrochloride, troglitazone or rosiglitazone, rosiglitazone maleate, or in each case the pharmaceutically acceptable salt of such a compound.
The preferred DDP-IV inhibitor is selected from 1-{2-[(5-cyanopyridin-2-yl)amino]ethylamino}acetyl-2 (S)-cyano-pyrrolidine dihydrochloride (DPP728) especially the dihydrochloride thereof, (S)-1-[(3-hydroxy-1-adamantyl)amino]acetyl-2-cyano-pyrrolidine (LAF237), L-threo-isoleucyl thiazolidine (compound code according to Probiodrug: P32/98 as described above), MK-0431, 3-(aminomethyl)-2-isobuthyl-1-oxo-4-phenyl-1,2-dihydro-6-isoquinolinecarboxamide and 2-{[3-(aminomethyl)-2-isobuthyl-4-phenyl-1-oxo-1,2-dihydro-6-isoquinolyl]oxy}acetamide and optionally in any case pharmaceutical salts thereof.
The preferred DDP-IV inhibitor is selected from (S)-1-[(3-hydroxy-1-adamantyl)amino]acetyl-2-cyano-pyrrolidine (LAF237) and MK-0431 and optionally in any case pharmaceutical salts thereof.

### EXAMPLE

Preparation of bilayered tablets (one layer of metformin and one layer comprising (the DPP-4 inhibitor and pioglitazone):

INGREDIENTS: Mg/tablet; Metformin Hydrochloride 500; Microcrystalline Cellulose 245; Sodium Carboxymethy; Cellulose 150; Hydroxypropyl methylcellulose 100; Magnesium Stearate; Hydroxypropyl methylcellulose E5 15.6; Seal Coat Polyethylene glycol 4000 4.8; Titanium Dioxide 2.4; Pioglitazone hydrochloride equiv. to pioglitazone (30 ma) 39.672 Pioglitazone; DPP-IV inhibitor 50; Lactose 80; layer Hydroxypropyl cellulose 2.4; Carboxymethyl cellulose calcium 3.6; Magnesium stearate 1.2; Purified water q.s.

### Procedure:

1. Metformin hydrochloride was milled and mixed with microcrystalline cellulose and sodium carboxymethyl cellulose. The blend was sieved.
2. Hydroxypropyl methylcellulose was separately sifted and mixed with the blend of; step 1 in a low shear mixer. The blend was then mixed with magnesium stearate and passed through roller compactor and then milled again to form granules.
3. Pioglitazone, DPP-IV inhibitor, lactose, hydroxypropyl cellulose and carboxymethylcellulose calcium were blended and granulated with purified water.
4. The wet mass of step 3 was granulated, dried and sifted.
5. The lubricated granules of metformin and (pioglitazone+DPP-IV inhibitor) were compressed into bilayer tablets using a rotary compression machine i.e. 1 layer of metformin and one layer comprising (the DPP-4 inhibitor and pioglitazone).

Further, it is contemplated that any single feature or any combination of optional features of the inventive variations described herein may be specifically excluded from the claimed invention and be so described as a negative limitation. Accordingly, it is not intended that the invention be limited, except as by the appended claims.

Any of the substances e.g. compounds or formulations, disclosed in the above mentioned patent documents or literature documents, are hereby included by reference.

What is claimed is:

1. A combination which comprises:
i) a dipeptidylpeptidase-IV (DPP-IV) inhibitor,
ii) one PPAR ANTIDIABETIC selected from thiazolidinediones (glitazones), non-glitazone type PPARγ agonists, PPARα agonists or dual PPARγ/PPARα agonists, and
iii) metformin,
or in each case the pharmaceutically acceptable salt of such a compound and optionally at least one pharmaceutically acceptable carrier; for simultaneous, separate or sequential use.

2. The combination according to claims 1, which is in the form of a fixed combination.

3. The combination according to claim 2, wherein the DPP-IV inhibitor, the PPAR ANTIDIABETIC and metformin are in the form of a conventional immediate release formulation.

4. The combination according to claim 2, wherein;
i) the DPP-IV inhibitor and the PPAR ANTIDIABETIC are in the form of a conventional immediate release formulation, and
ii) metformin is in an extended release formulation.

5. The combination according to claim 1, wherein the PPAR ANTIDIABETIC is selected from Compound L, pioglitazone, pioglitazone hydrochloride, troglitazone or rosiglitazone, rosiglitazone maleate, or in each case the pharmaceutically acceptable salt of such a compound.

6. The combination according to claim 1, wherein the DDP-IV inhibitor is selected from 1-{2-[(5-cyanopyridin-2-yl)amino]ethylamino}acetyl-2 (S)-cyano-pyrrolidine dihydrochloride (DPP728) especially the dihydrochloride thereof, (S)-1-[(3-hydroxy-1-adamantyl)amino]acetyl-2-cyano-pyrrolidine (LAF237), L-threo-isoleucyl thiazolidine (compound code according to Probiodrug: P32/98 as described above), MK-0431, 3-(aminomethyl)-2-isobuthyl-1-oxo-4-phenyl-1,2-dihydro-6-isoquinolinecarboxamide and 2-{[3-(aminomethyl)-2-isobuthyl-4-phenyl-1-oxo-1,2-dihydro-6-isoquinolyl]oxy}acetamide and optionally in any case pharmaceutical salts thereof.

7. The combination according to claim 1, wherein the DDP-IV inhibitor is (S)-1-[(3-hydroxy-1-adamantyl)amino]acetyl-2-cyano-pyrrolidine (LAF237) and the PPAR ANTIDIABETIC is selected from Compound L, pioglitazone or rosiglitazone,
or in each case the pharmaceutically acceptable salt of such a compound.

8. (canceled)

9. A method of treating a condition mediated by DPP-IV, comprising
administering to a warm-blooded animal in need thereof jointly therapeutically effective amounts of (S)-1-[3-hydroxy-1-adamantyl)amino]acetyl-2-cyano-pyrrolidine (LAF237) or a pharmaceutical salt thereof, in combination with;
i) metformin or a salt thereof, and
ii) pioglitazone or rosiglitazone or in any case pharmaceutical salts thereof.

10. (canceled)

11. (canceled)

12. The method according to claim 9, wherein a daily dosage 100 mg of LAF237 or a pharmaceutical salt thereof is administered once a day, preferably with or before the meal.

13. The method according to claim 9, wherein a daily dosage 100 mg of LAF237 or a pharmaceutical salt thereof is administered, and divided in two separate administrations of 50 mg.

14. The method according to claim 9, wherein a daily dosage of 15, 30 or 45 mg of pioglitazone or a pharmaceutical salt thereof is administered.

15. The method according to claim 9, wherein a daily dosage of 15 or 30 mg of pioglitazone or a pharmaceutical salt thereof is administered once daily.

16. The method according to claim 9, wherein a daily dosage of 4 or 8 mg of rosiglitazone or rosiglitazone maleate or any pharmaceutical salt thereof is administered.

17. The method according to claim 9, wherein a daily dosage of 4 or 8 mg of rosiglitazone or rosiglitazone maleate or any pharmaceutical salt thereof is administered as a single dose or divided twice daily.

18. The method according to claim 9, wherein a daily dosage of 4 or 8 mg of rosiglitazone or rosiglitazone maleate is administered.

19. The method according to claim 9, wherein a daily dosage of 250, 500, 750, 850, 1000, 1500, 2000 or 2550 mg of metformin or a pharmaceutical salt thereof is administered.

19. (canceled)

20. The method according to claim 9, wherein a daily dosage of 2000 mg of metformin or a salt thereof is administered, and divided in 2 administrations of 1000 mg a day.

21. The method according to claim 9, wherein a daily dosage of 1500 mg of metformin or a salt thereof is administered, and divided in 2 administrations of 750 mg a days or 3 administrations of 500 mg a day.

22. The method according to claim 9, wherein a daily dosage of 1000 mg of metformin or a salt thereof is administered, once a day or divided in 2 administrations of 500 mg a day.

23. The method according to claim 9, wherein a daily dosage of 750 mg of metformin or a salt thereof is administered, once a day, or divided in 3 administrations of 250 mg a day.

24. The method according to claim 9, wherein a daily dosage of 500 mg of metformin or a salt thereof is administered, once a day or divided in 2 administrations of 250 mg a day.

25. The method according to claim 9, wherein at least one tablet is administered in the form of a fixed combination comprising;
i) LAF237 and metformin or in any case a salt thereof,
ii) pioglitazone and metformin or in any case a salt thereof,
iii) rosiglitazone and metformin or in any case a salt thereof,
iv) LAF237 and pioglitazone or in any case a salt thereof,
v) LAF237 and rosiglitazone or in any case a salt thereof,
vi) LAF237 and pioglitazone and metformin or in any case a salt thereof, or
vii) LAF237 and rosiglitazone and metformin or in any case a salt thereof.

26. The method according to claim 9, wherein a daily dosage of 2550 mg of metformin or a salt thereof is administered, and divided in 3 administrations of 850 mg a day.
